(19) United States
(12) Patent Application Publication
(10) Pub. No.: US 2009/0200925 A1
(43) Pub. Date: Aug. 13, 2009

(54) ORGANIC ELECTROLUMINESCENT ELEMENT, LIGHTING DEVICE AND DISPLAY DEVICE

(75) Inventors: Mitsuyoshi Naito, Tokyo (JP); Hiroshi Kita, Tokyo (JP); Yoshiyuki Suzuri, Tokyo (JP); Aki Tsuji, Kyoto (JP)

Correspondence Address: LUCAS & MERCANTI, LLP, 475 PARK AVENUE SOUTH, 15TH FLOOR, NEW YORK, NY, 10016, US

(73) Assignee: KONICA MINOLTA HOLDINGS, INC., Tokyo (JP)

(21) Appl. No.: 12/304,122

(22) PCT Filed: Jun. 7, 2007
(86) PCT No.: PCT/JP2007/061542

§ 371 Date: Dec. 10, 2008

(30) Foreign Application Priority Data
Jun. 13, 2006 (JP) 2006163223

(51) Int. Cl. H01J 1/63

(52) U.S. Cl. 313/504

(57) ABSTRACT

Provided is a means to obtain long lifetime of an organic EL element, and a long lifetime lighting device and a long lifetime display device utilizing the means are further provided. Disclosed is an organic electroluminescent element comprising an emission layer containing a host material and a dopant material, and a hole transport layer, provided between cathode and anode facing each other, wherein an intermediate layer is provided on an anode side of the emission layer so as to be brought into contact with the emission layer, and the hole transport layer is provided on the anode side of the intermediate layer, satisfying the following Formulae E1<E2≦E3 and E2>E4, where E1 represents an ionization potential of a hole transport material, E2 represents an ionization potential of an intermediate material, E3 represents an ionization potential of the host material, and E4 represents an ionization potential of the dopant material.

### TECHNICAL FIELD

The present invention relates to an organic electroluminescent element (hereinafter, referred to also as an organic EL element), a display device and a lighting device.

### BACKGROUND

Attention is focused on an organic EL element as a lighting device or a display device, because high luminance emission can be produced at low voltage.

The organic electroluminescent element is constituted in such a manner that an emission layer containing light emitting compounds is sandwiched between a cathode and an anode. In the above element, electrons and holes are injected into the emission layer and are subjected to recombination, whereby exciton is generated. During deactivation of the resulting exciton, light (fluorescence or phosphorescence) is emitted.

Various studies concerning long lifetime of an organic EL element have been done so far since they are of significance for practical use and various applications. Various methods to improve long lifetime have been proposed, and studies have been made from both sides of sealing and fabrication. The long lifetime of an organic EL element are to be achieved via overall studies from both sides of these, but further studies have still been made continuously. As an attempt thereof, it is reported that in the organic EL element possessing an emission layer containing a dopant molecule, the long lifetime is obtained by controlling a luminescence region within the emission layer, that is, the dopant molecule content in the emission layer, and by producing luminescence principally in an inner region of the emission layer while inhibiting luminescence at an emission layer interface (refer to Patent Document 1, for example).

However, long lifetime of the organic EL element has still been demanded, further specific means and techniques are desired to be developed.

Patent Document 1: Japanese Patent O.P.I. Publication No. 2005-108730

### DISCLOSURE OF THE INVENTION

### Problems to be Solved by the Invention

The present invention has been made on the basis of the above-described situation, and it is an object of the present invention to provide a means to obtain long lifetime of an organic EL element, and further to provide a long lifetime lighting device as well as a long lifetime display device employing the means.

### Means to Solve the Problems

The above-described object of the present invention is accomplished by the following structures.

(Structure 1) An organic electroluminescent element comprising an emission layer containing a host material and a dopant material, and a hole transport layer, provided between a cathode and an anode facing each others wherein an intermediate layer is provided on an anode side of the emission layer so as to be brought into contact with the emission layer, and the hole transport layer is provided on the anode side of the intermediate layer, the organic electroluminescent element satisfying the following Formulae (2) and (3):

E1<E2≦E3 Formula (2)

E2>E4 Formula (3)

wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material.

(Structure 2) An organic electroluminescent element comprising an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is provided on the anode side of the intermediate layer so as to be brought into contact with the intermediate layer, the organic electroluminescent element satisfying the following Formula

μe>μh Formula (1)

wherein μe and μh represent electron mobility and hole mobility of the host material, respectively, and further satisfying the following Formulae (2) and (3):

E1<E2≦E3 Formula (2)

E2>E4 Formula (3)

wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material.

(Structure 3) The organic electroluminescent element of Structure 1 or 2, wherein the dopant material produces phosphorescence.

(Structure 4) The organic electroluminescent element of any one of Structures 1-3, wherein the dopant material has a triplet excitation energy of at least 2.58 eV.

(Structure 5) The organic electroluminescent element of any one of Structures 1-4, wherein the dopant material has an ionization potential E4 of 5.3 eV or less.

(Structure 6) The organic electroluminescent element of any one of Structures 1-5, wherein the intermediate material has a triplet excitation energy of at least 2.58 eV.

(Structure 7) The organic electroluminescent element of any one of Structures 1-6, wherein the intermediate material and the host material are the same in compound.

(Structure 8) The organic electroluminescent element of any one of Structures 1-7, wherein the intermediate layer has a thickness of 1-20 nm.

(Structure 9) The organic electroluminescent element of any one of Structures 1-8, satisfying the following Formula (4):

0.001<L2/(L1+L2+L3)<0.2 Formula (4)

wherein L1 represents thickness of the hole transport layer, L2 represents thickness of the intermediate layer, and L3 represents thickness of the emission layer.

(Structure 10) The organic electroluminescent element of any one of Structures 1-9, wherein the host material comprises any one of a carbazole ring, a carboline ring and a triaryl amine structure.

(Structure 11) The organic electroluminescent element of any one of Structures 1-10, wherein the dopant material comprises a compound having a partial structure represented by the following Formula (1):

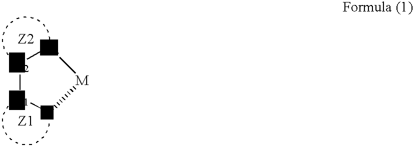

wherein X₁, X₂ and X₃ each represent a carbon atom or a nitrogen atom; Z1 represents a residue to form a 5-member aromatic heterocycle; Z2 represents a 6-member aromatic ring, a 5-member aromatic heterocycle or a 6-member aromatic heterocycle; and M represents Ir or Pt.

(Structure 12) The organic electroluminescent element of any one of Structures 1-11, wherein the dopant material comprises a compound having a partial structure represented by the following Formula (6):

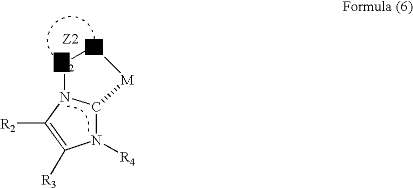

wherein X₂ and X₃ each represent a carbon atom or a nitrogen atom; R₂, R₃ and R₄ each represent a hydrogen atom or a substituent; Z2 represents a 6-member aromatic ring, a 5-member aromatic heterocycle or a 6-member aromatic heterocycle; and M represents Ir or Pt.

(Structure 13) The organic electroluminescent element of any one of Structures 1-12, wherein the intermediate material comprises any one of a carbazole ring, a carboline ring and a triaryl amine structure.

(Structure 14) An organic electroluminescent element comprising an emission layer containing a host material and a dopant material, and a hole transport layer, provided between a cathode and an anode facing each other,

wherein an intermediate layer is provided on an anode side of the emission layer so as to be brought into contact with the emission layer, and the hole transport layer is provided on the anode side of the intermediate layer, the organic electroluminescent element satisfying the following Formula (5):

μ1>μ2 Formula (5)

wherein μ1 represents hole mobility of the hole transport material, and μ2 represents hole mobility of an intermediate material constituting the intermediate layer.

(Structure 15) An organic electroluminescent element comprising an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is provided on the anode side of the intermediate layer, the organic electroluminescent element satisfying the following Formula (1):

μe>μh Formula (1)

wherein μe and μh represent electron mobility and hole mobility of the host material, respectively, and further satisfying the following Formula (5):

μ1>μ2 Formula (5)

wherein μ1 represents hole mobility of the hole transport material, and μ2 represents hole mobility of an intermediate material constituting the intermediate layer.

(Structure 16) The organic electroluminescent element of Structure 14 or 15, wherein the dopant material produces phosphorescence.

(Structure 17) The organic electroluminescent element of any one of Structures 14-16, wherein the dopant material has a triplet excitation energy of at least 2.58 eV.

(Structure 18) The organic electroluminescent element of any one of Structures 14-17, wherein the dopant material has an ionization potential E4 of 5.3 eV or less.

(Structure 19) The organic electroluminescent element of any one of Structures 14-18, wherein the intermediate material has a triplet excitation energy of at least 2.58 eV.

(Structure 20) The organic electroluminescent element of any one of Structures 14-19, wherein the intermediate material and the host material are the same in compound.

(Structure 21) The organic electroluminescent element of any one of Structures 14-20, wherein the intermediate layer has a thickness of 1-20 nm.

(Structure 22) The organic electroluminescent element of any one of Structures 14-21, satisfying the following Formula (4):

0.001<L2/(L1+L2+L3)<0.2 Formula (4)

wherein L1 represents thickness of the hole transport layer, L2 represents thickness of the intermediate layer, and L3 represents thickness of the emission layer.

(Structure 23) The organic electroluminescent element of any one of Structures 14-22, wherein the host material comprises any one of a carbazole ring, a carboline ring and a triaryl amine structure.

(Structure 24) The organic electroluminescent element of any one of Structures 14-23, wherein the dopant material comprises a compound having a partial structure represented by the following Formula (1):

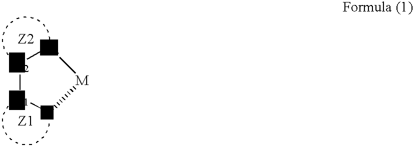

wherein X₁, X₂ and X₃ each represent a carbon atom or a nitrogen atom; Z1 represents a residue to form a S-member aromatic heterocycle; Z2 represents a 6-member aromatic ring, a 5-member aromatic heterocycle or a 6-member aromatic heterocycle; and M represents Ir or Pt.

(Structure 25) The organic electroluminescent element of any one of Structures 14-24, wherein the dopant material comprises a compound having a partial structure represented by the following Formula (6):

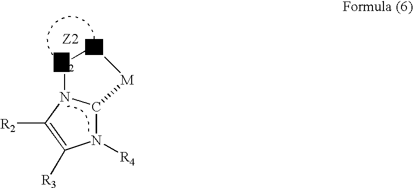

wherein X₂ and X₃ each represent a carbon atom or a nitrogen atom; R₂, R₃ and R₄ each represent a hydrogen atom or a substituent; Z2 represents a 6-member aromatic ring, a 5-member aromatic heterocycle or a 6-member aromatic heterocycle; and M represents Ir or Pt.

(Structure 26) The organic electroluminescent element of any one of Structures 14-25, wherein the intermediate material comprises any one of a carbazole ring, a carboline ring and a triaryl amine structure.

(Structure 27) An organic electroluminescent element comprising an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is provided on the anode side of the intermediate layer, the organic electroluminescent element satisfying the following Formulae (2) and (3):

E1<E2≦E3 Formula (2)

E2>E4 Formula (3)

wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material, and further satisfying the following Formula (5):

μ1>μ2 Formula (5)

wherein μ1 represents hole mobility of the hole transport material, and μ2 represents hole mobility of an intermediate material constituting the intermediate layer.

(Structure 28) An organic electroluminescent element comprising an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is provided on the anode side of the intermediate layer so as to be brought into contact with the intermediate layers the organic electroluminescent element satisfying the following Formula (1):

μe>μh Formula (1)

wherein μe and μh represent electron mobility and hole mobility of the host material, respectively; also satisfying the following Formulae (2) and (3):

E1<E2≦E3 Formula (2)

E2>E4 Formula (3)

wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material; and further satisfying the following Formula (5):

μ1>μ2 Formula (5)

wherein μ1 represents hole mobility of the hole transport material, and μ2 represents hole mobility of an intermediate material constituting the intermediate layer.

(Structure 29) The organic electroluminescent element of Structure 27 or 28, wherein the dopant material produces phosphorescence.

(Structure 30) The organic electroluminescent element of any one of Structures 27-29, wherein the dopant material has a triplet excitation energy of at least 2.58 eV.

(Structure 31) The organic electroluminescent element of any one of Structures 27-30, wherein the dopant material has an ionization potential E4 of 5.3 eV or less.

(Structure 32) The organic electroluminescent element of any one of Structures 27-31, wherein the intermediate material has a triplet excitation energy of at least 2.58 eV.

(Structure 33) The organic electroluminescent element of any one of Structures 27-32, wherein the intermediate material and the host material are the same in compound.

(Structure 34) The organic electroluminescent element of any one of Structures 27-33, wherein the intermediate layer has a thickness of 1-20 nm.

(Structure 35) The organic electroluminescent element of any one of Structures 27-34, satisfying the following Formula (4):

0.001<L2/(L1+L2+L3)<0.2 Formula (4)

wherein L1 represents thickness of the hole transport layer, L2 represents thickness of the intermediate layer, and L3 represents thickness of the emission layer.

(Structure 36) The organic electroluminescent element of any one of Structures 27-35, wherein the host material comprises any one of a carbazole ring, a carboline ring and a triaryl amine structure.

(Structure 37) The organic electroluminescent element of any one of Structures 27-36, wherein the dopant material comprises a compound having a partial structure represented by the following Formula (1):

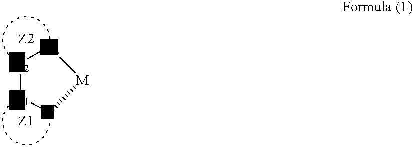

wherein X₁, X₂ and X₃ each represent a carbon atom or a nitrogen atom; Z1 represents a residue to form a 5-member aromatic heterocycle; Z2 represents a 6-member aromatic ring, a 5-member aromatic heterocycle or a 6-member aromatic heterocycle; and M represents Ir or Pt.

(Structure 38) The organic electroluminescent element of any one of Structures 27-37, wherein the dopant material comprises a compound having a partial structure represented by the following Formula (6):

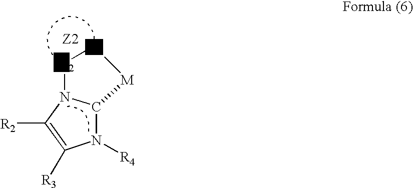

wherein X₂ and X₃ each represent a carbon atom or a nitrogen atom; R₂, R₃ and R₄ each represent a hydrogen atom or a substituent; Z2 represents a 6-member aromatic ring, a 5-member aromatic heterocycle or a 6-member aromatic heterocycle; and M represents It or Pt.

(Structure 39) The organic electroluminescent element of any one of Structures 27-38, wherein the intermediate material comprises any one of a carbazole ring, a carboline ring and a triaryl amine structure.

(Structure 40) A lighting device comprising the organic electroluminescent element of any one of Structures 1-39.

(Structure 41) A display device comprising the organic electroluminescent element of any one of Structures 1-39.

### EFFECT OF THE INVENTION

A long lifetime organic EL element can be obtained in the present invention, and a long lifetime lighting device as well as a long lifetime display device can be provided by utilizing the organic EL element.

### BRIEF DESCRIPTION OF THE DRAWINGS

FIG. 1 is a schematic illustration diagram showing an example of ionization potential of each of a hole transport material, a host material and a dopant material contained in an emission layer in an organic EL element.

FIG. 2 is a schematic illustration diagram showing ionization potential of each material in cases where an intermediate layer is provided.

### DESCRIPTION OF THE PREFERRED EMBODIMENTS

The preferred embodiments of the present invention will be explained below, but the present invention is not limited thereto.

In the present invention, disclosed is an organic electroluminescent element possessing at least an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is further provided on the anode side of the intermediate layer so as to be brought into contact with the intermediate layer, the organic electroluminescent element having a layered structure of the hole transport layer, the intermediate layer and the emission layer in order from the anode side.

In the present invention, the relationship between electron mobility μe and hole mobility μh preferably satisfies the following Formula (1).

μe>μh Formula (1)

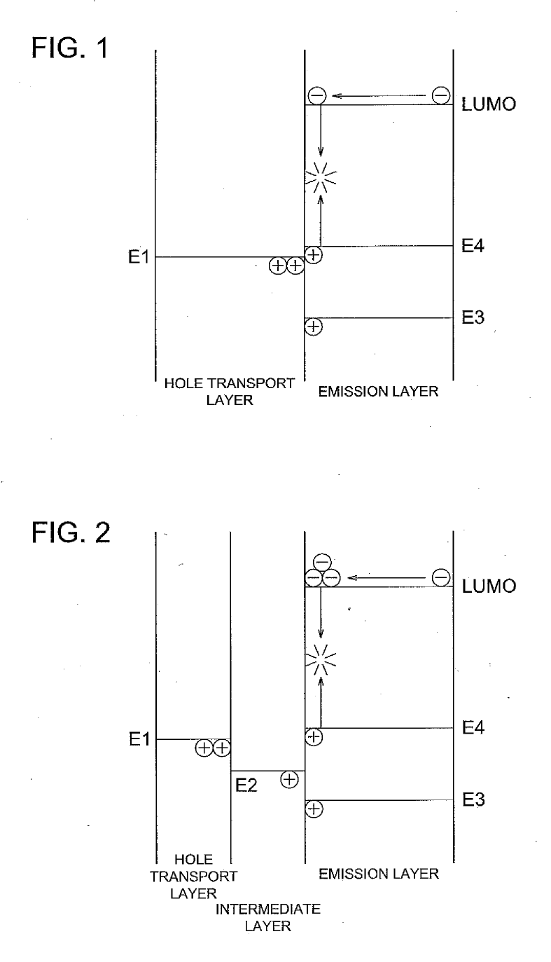

FIG. 1 is a schematic illustration diagram showing the relationship of the ionization potential of each of a hole transport material, a host material and a dopant material contained in an emission layer (E1, E3 and E4, respectively) in an organic EL element in the case of no intermediate layer provided. In the case of no intermediate layer (FIG. 1), when holes are injected from a hole transport layer to a host material and electron mobility μe of the host material is larger than hole mobility of that, injected holes can not be moved onto the cathode side, and are trapped in the dopant material, whereby luminescence is to be produced when electrons are injected into the dopant material. Since a duration of trapping holes in the dopant material becomes longer, the dopant material is deteriorated, and as the result, lifetime of the organic EL element becomes shorter.

FIG. 2 is a schematic illustration diagram showing the relationship of the ionization potential of a hole transport material, an intermediate (layer) material, a host material and a dopant material contained in an emission layer in an organic EL element of the present invention, in cases where the intermediate layer is provided. In the present invention, the intermediate layer is inserted between the hole transport layer and the emission layer (FIG. 2), and is to be selected in such a way that the relationship among ionization potential E1 of the hole transport material constituting the hole transport layer, ionization potential E2 of the intermediate material constituting the foregoing intermediate layer, ionization potential E3 of the foregoing host material and ionization potential E4 of the foregoing dopant material E4 satisfies the following Formulae (2) and (3).

E1<E2≦E3 Formula (2)

E2>E4 Formula (3)

By using the above, it was found out that hole-trapping time was controlled with the dopant material in the emission layer to suppress degradation of the dopant material, whereby the longer lifetime was achieved.

Accordingly, one of the preferred embodiments is of the case where the relationship among ionization potential E1 of the hole transport material constituting the hole transport layer, ionization potential E2 of the intermediate material constituting the foregoing intermediate layer, ionization potential E3 of the foregoing host material and ionization potential E4 of the foregoing dopant material E4 satisfies foregoing Formulae (2) and (3).

When a dopant material producing fluorescence is compared with a dopant material producing phosphorescence, the dopant material producing phosphorescence is more preferred as the dopant material. Since the dopant material producing phosphorescence is stably lower in a hole-trapping situation than the dopant material producing fluorescence, the dopant material producing phosphorescence is more effective for longer lifetime than the dopant material producing fluorescence.

The dopant material has a triplet excitation energy of at least 2.58 eV, that is, one having an emission wavelength in the blue region is preferable. The larger the triplet excitation energy of the dopant material, the more the dopant material is degraded. Therefore, the dopant material having an emission wavelength in the blue region, which has a triplet excitation energy of at least 2.58 eV is effective for longer lifetime.

The dopant material having an ionization potential E2 of 5.3 eV or less is preferable. The dopant material is easy to be degraded since one having an ionization potential E2 of 5.3 eV or less is easy to trap holes. Therefore, the dopant material having an ionization potential E2 of 5.3 eV or less is effective for longer lifetime.

An intermediate layer of the present invention may be brought into contact with a hole transfer layer, and an emission layer may be provided between the intermediate layer and the hole transfer layer.

As an intermediate material constituting the intermediate layer, one having a triplet excitation energy of at least 2.58 eV is more preferable. That is, since the intermediate material having a triplet excitation energy of at least 2.58 eV is possible to trap the excitation energy in a dopant, the intermediate material having a triplet excitation energy of at least 2.58 eV can improve emission efficiency.

The intermediate material and the host material are preferably the same in compound. By using this, still longer lifetime can be designed to be obtained.

The intermediate layer preferably has a thickness L2 of 1-20 nm. The intermediate layer having a thickness L2 of 1-20 nm is effective for longer lifetime.

The intermediate layer having a thickness L2 of 5-10 nm is more preferable. By using this, still longer lifetime can be designed to be obtained.

Further, it is preferable that the relationship among thickness L1 of the hole transport layer, thickness L2 of the intermediate layer and thickness L3 of the emission layer is of one satisfying the following Formula (4).

0.001<L2/(L1+L2+L3)<0.2 Formula (4)

By using this, still longer lifetime can be designed to be obtained.

As one of the preferred embodiments of the present invention, disclosed is an organic electroluminescent element possessing at least an emission layer containing a host material and a dopant material, and a hole transport layer, provided between a cathode and an anode facing each other, wherein an intermediate layer is provided on an anode side of the emission layer so as to be brought into contact with the emission layer, and the hole transport layer is provided on the anode side of the intermediate layer so as to be brought into contact with the intermediate layer, the organic electroluminescent element having a layered structure of the hole transport layer, the intermediate layer and the emission layer in order from the anode side, and satisfying the following Formula (5):

μ1>μ2 Formula (5)

wherein μ1 represents hole mobility of the hole transport material, and μ2 represents hole mobility of the intermediate material. By using this, it was found out that hole-trapping time was controlled with the dopant material in the emission layer to suppress degradation of the dopant material, whereby the longer lifetime was achieved.

That is, traveling time of holes is delayed by inserting an intermediate layer satisfying the above-described condition between the hole transport layer and the emission layer. In other words, in comparison to no intermediate layer provided, the number of holes injected into a dopant material is controlled in this case since the holes run through the intermediate layer.

In this case, when electron mobility μe of the host material is larger than hole mobility μh of the host material, a large amount of electrons injected into the host material from the cathode side are to be present in the region in the emission layer on the intermediate layer side. As a result, when the above-described layer is inserted, and holes injected into the dopant material from the intermediate layer are trapped in the dopant material, luminescence can be produced in a short period of time.

Accordingly, what the relationship between electron mobility μe of the host material and hole mobility μh of the host material satisfies the following Formula (1) is preferable in view of acquisition of further longer lifetime thereof.

μe>μh Formula (1)

Further, it is more preferable that Formulae (2), (3) and (5) are satisfied at the same time in view of acquisition of longer lifetime thereof, and still more preferable that the above-described formulae together with Formula (1) are satisfied at the same time.

That is, utilized is an organic electroluminescent element comprising an emission layer containing a host material and a dopant material, and a hole transport layer provided between a cathode and an anode facing each other, wherein an intermediate layer is provided on an anode side of the emission layer so as to be brought into contact with the emission layer, and the hole transport layer is provided on the anode side of the intermediate layer, the organic electroluminescent element satisfying the following Formulae (2) and (3):

E1<E2≦E3 Formula (2)

E2>E4 Formula (3)

wherein ionization potential of a hole transport material constituting the hole transport layer is represented by E1, ionization potential of an intermediate material constituting the intermediate layer is represented by E2, ionization potential of the host material is represented by E3, and ionization potential of the dopant material is represented by E4, and further satisfying the following Formula (5):

μ1>μ2 Formula (5)

wherein hole mobility of the hole transport material is represented by μ1 and hole mobility of the intermediate material is represented by μ2, in order to control hole trapping time by the dopant material in the emission layer and to inhibit deterioration of the dopant material, and thus the longer lifetime can be achieved.

Also, similarly in this case, it is preferable that longer lifetime thereof can further be obtained by satisfying the following Formula (1):

μe>μh Formula (1)

wherein electron mobility of the host material is represented by μe, and the hole mobility is represented by μh.

Constituent layers in the organic EL element of the present invention will further be described in detail. Preferable specific examples concerning the constituent layers in the organic EL element of the present invention are shown below, but the present invention is not limited thereto.

(1) anode/hole transport layer/intermediate layer/emission layer/cathode

(2) anode/hole transport layer/intermediate layer emission layer/electron transport layer/cathode

(3) anode/hole transport layer/intermediate layer/emission layer/hole blocking layer/electron transport layer/cathode

(4) anode/hole transport layer/intermediate layer/emission layer/electron transport layer/cathode

(5) anode/hole transport layer/intermediate layer/electron blocking layer/emission layer/hole blocking layer/electron transport layer/cathode

(6) anode/anode buffer layer/hole transport layer/intermediate layer/electron blocking layer/emission layer/hole blocking layer/electron transport layer/cathode buffer layer/cathode

(7) anode/anode buffer layer/hole transport layer/emission layer/intermediate layer/electron blocking layer hole blocking layer/electron transport layer/cathode buffer layer/cathode

### <<Anode>>

As an anode fitted into the organic EL element, preferably employed are those which employ, as electrode materials, metals, alloys, electrically conductive compounds, and mixtures thereof, which exhibit a relatively high work function (at least 4 eV). Specific examples of such electrode materials include metals such as Au, and electrically conductive transparent materials such as Cut, indium tin oxide (ITO), SnO₂, or ZnO. Further employed may be IDIXO (In₂O₃-ZNO) which enables formation of an amorphous, transparent, and electrically conductive film.

The anode may be formed in such a manner that a thin film is formed via methods such as vapor deposition or sputtering, employing these electrode materials, and the desired shaped pattern is formed via a photolithographic method. Further, when pattern accuracy is not strongly needed (roughly at least 100 μm), a pattern may be formed via the desired shaped mask during vapor deposition or sputtering of the above electrode materials.

When luminescence is output from this anode, it is desirable that transmittance is at least 10%, and it is preferable that sheet resistance as the anode is at most several hundred Ω/□. Further, the electrode film thickness, depending on materials, is commonly in the range of 10-1000 nm, but is preferably in the range of 10-200 nm.

### <<Cathode>>

On the other hand, as a cathode preferably employed are those which employ, as electrode materials, metal (called electron injecting metals), alloys, electrically conductive compounds, and mixtures thereof, which exhibit a relatively low work function (at most 4 eV). Specific examples of such electrode materials include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, and rare earth metals.

Of these, in view of electron injection capability and resistance to oxidation, suitable are mixtures of an electron injecting metal and a second metal which is stable and exhibits a higher work function than that of the above metal, such as a magnesium/silver mixture, a magnesium/aluminum mixture, a magnesium/indium mixture, an aluminum/aluminum oxide (Al₂O₃) mixture, a lithium/aluminum mixture, or aluminum.

It is possible to prepare the cathode via formation of a thin film of the above electrode materials, employing methods such as vapor deposition or sputtering. It is preferable that sheet resistance as the cathode is at most a few hundred Ω/□. Further, the film thickness is commonly selected to be in the range of 10 nm-5 μm, but is preferably selected to be in the range of 50-200 nm. In addition, in order to transmit emitted light, it is advantageous that either the anode or the cathode is transparent or translucent to enhance emission luminance.

Further, it is possible to prepare a transparent or translucent cathode in such a manner that after preparing the above metal film, of a thickness of 1-20 nm, on the cathode, conductive transparent materials, listed in the description of the anode, are applied onto the above film. By applying the above, it is possible to prepare an element in which both the anode and the cathode exhibit transparency.

The emission layer of the present invention containing a host material and a dopant material is a layer in which electrons and holes injected from the cathode side or the anode side to the host material are recombined to produce luminescence, and the emitting portion may be within the emission layer or at the interface between the emission layer and the adjacent layer.

A host material and a dopant material of the present invention constitute an emission layer, and a material having a larger mixed ratio is the host material, and a material having a smaller mixed ratio is the dopant material.

The host compound contained in the emission layer in the organic EL element of the present invention preferably has a content of at least 20% by weight, based on compounds contained in the emission layer

The host compound contained in the emission layer in the organic EL element of the present invention is a compound in which phosphorescent emission has a phosphorescent quantum yield of less than 0.1 at room temperature (25° C.), and preferably has a phosphorescent quantum yield of less than 0.01 at room temperature (25° C.).

The above-described phosphorescent quantum yield can be measured with a method described in the 4^{th} edition “Jikken Kagaku Koza 7”, Bunko II, page 398 (1992) published by Maruzen. Measurement of the phosphorescent quantum yield can be conducted in a solution employing various kinds of solvents, but in the case of a phosphorescence emitter of the present invention, the above-described phosphorescent quantum yield of at least 0.01 may be achieved in any of arbitrary solvents

Further, on the other hand, the phosphorescent dopant is a compound in which luminescence from the excited triplet is observed, which is specifically a compound by which phosphorescence is produced at room temperature (25° C.), and is defined as a compound having a phosphorescent quantum yield of at least 0.01 at 25° C., but preferable is a phosphorescent quantum yield of at least 0.1 at 25° C.

In the present invention, the host material is an organic compound containing any one of a carbazole ring, a carboline ring and a triaryl amine structure. In the present invention, examples of compounds containing a carbazole ring, a carboline ring (also called an azacarbazole ring, which represents one in which one of carbon atoms constituting the foregoing carbazole ring is substituted by a nitrogen atom) or a triaryl amine structure utilized as the host material are shown below, but the present invention is not limited thereto.

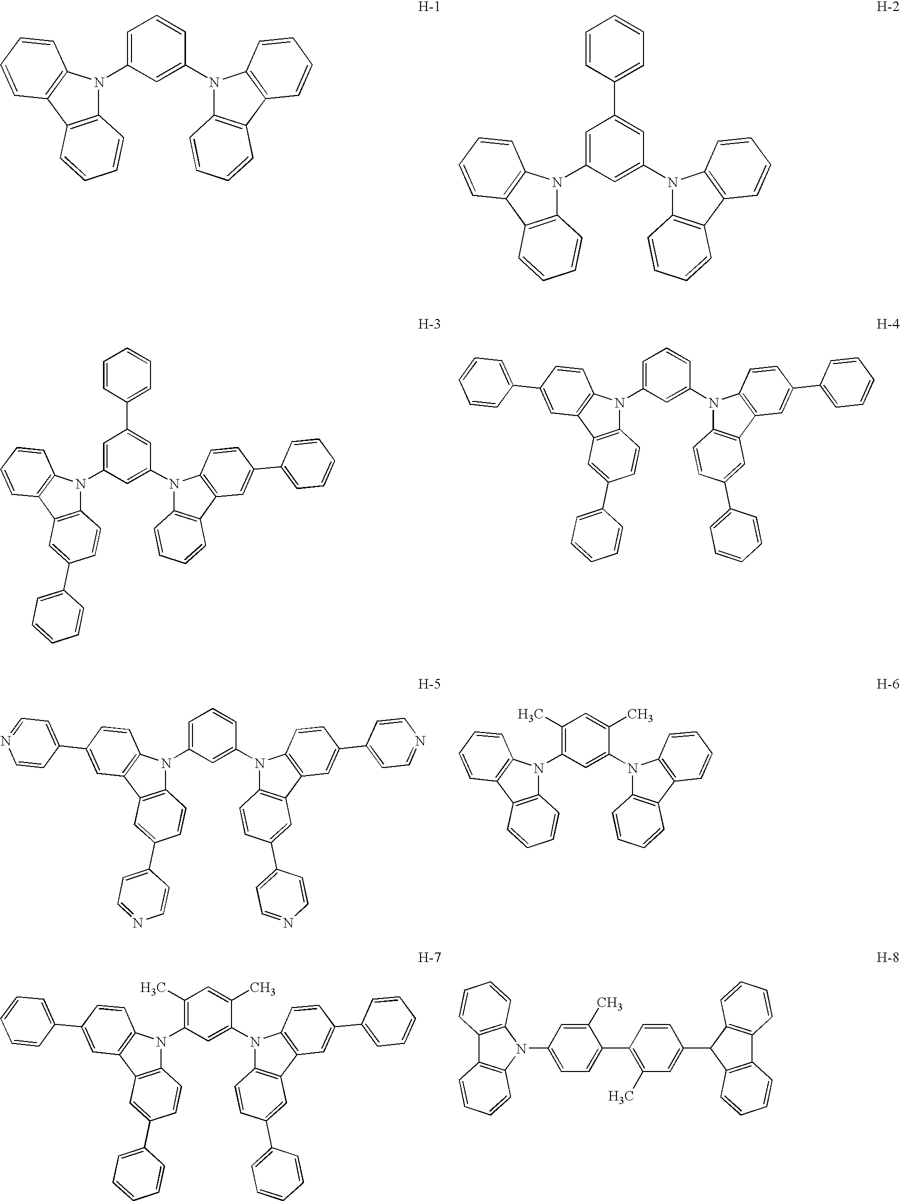

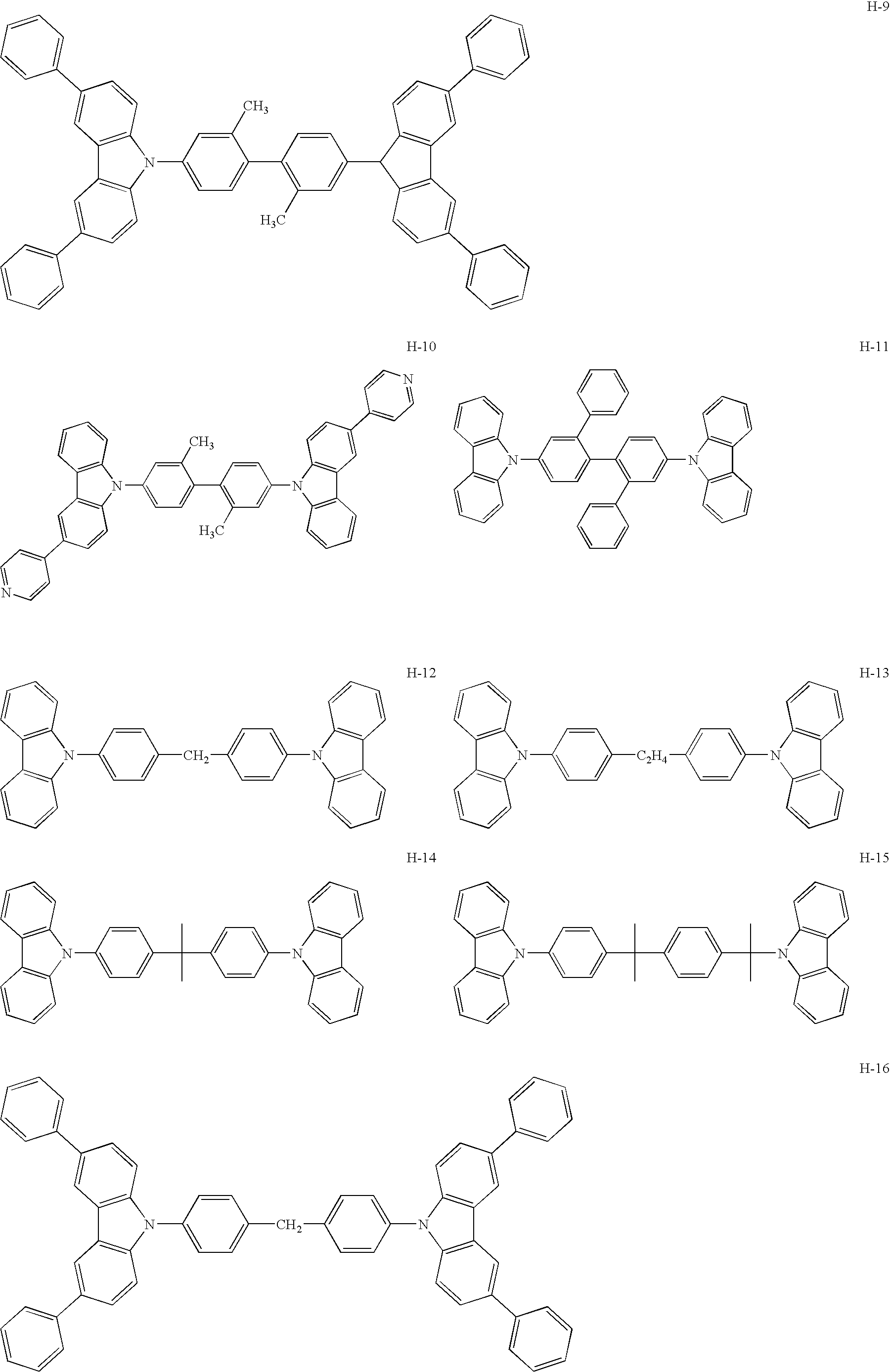

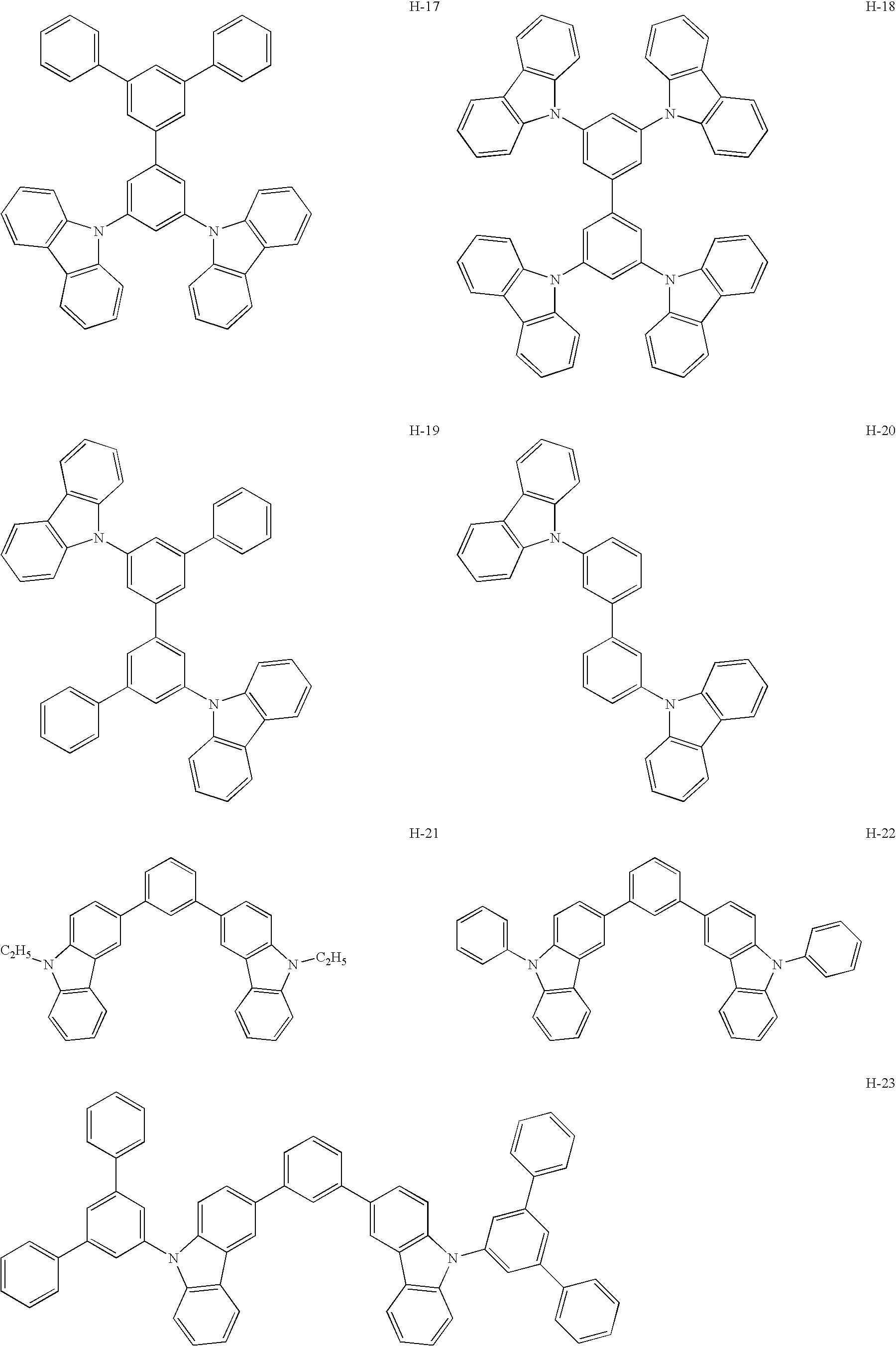

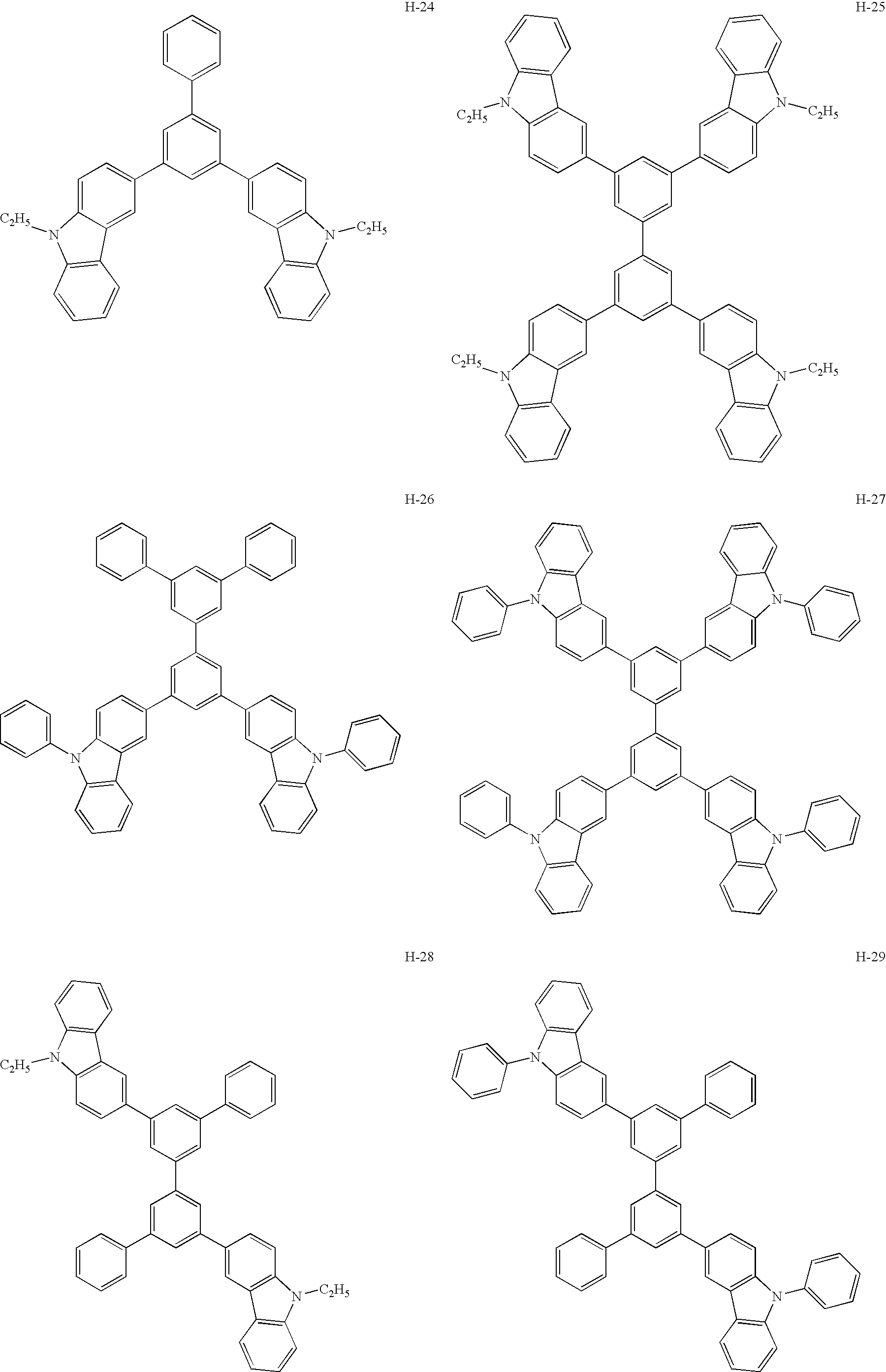

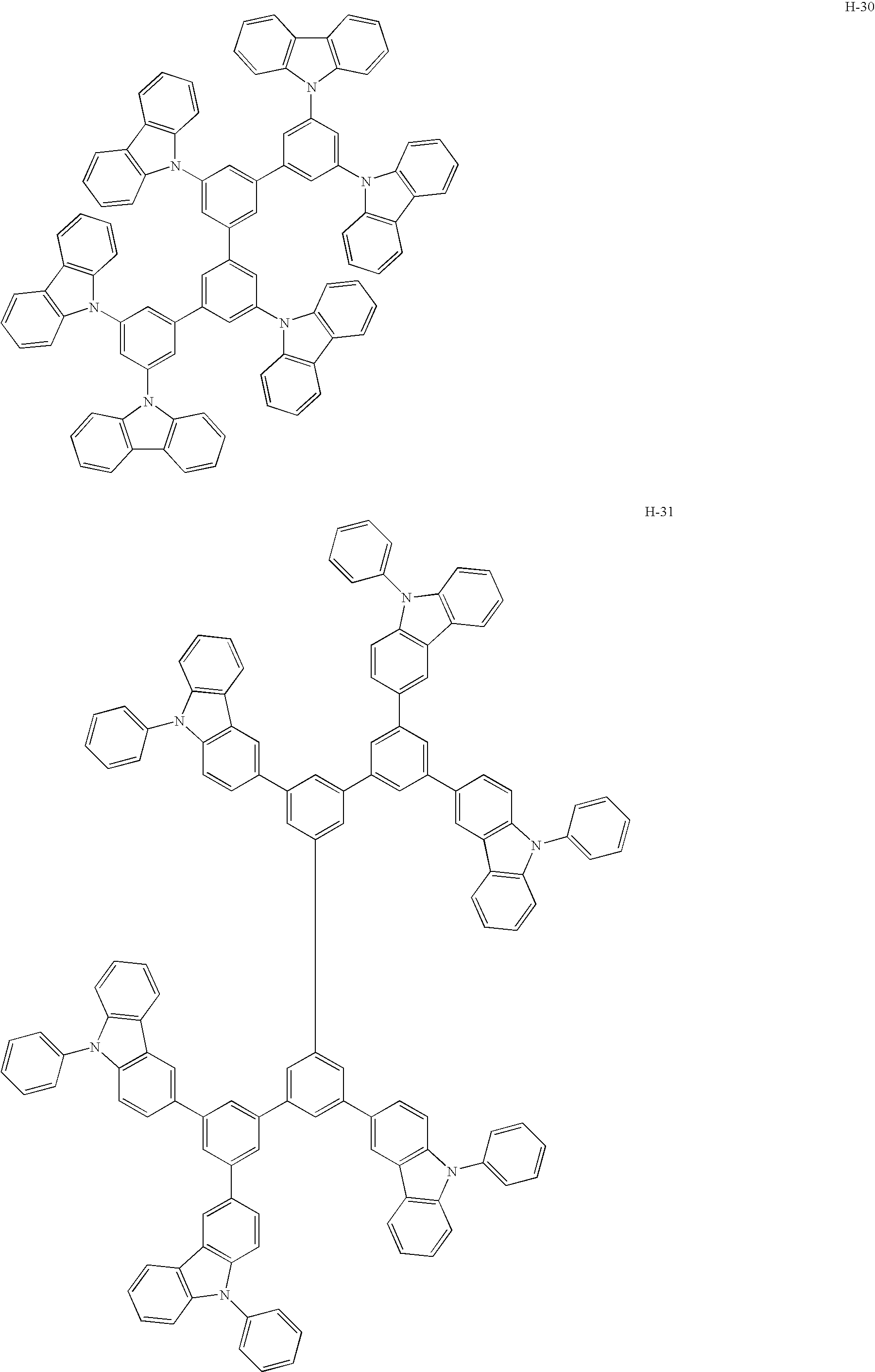

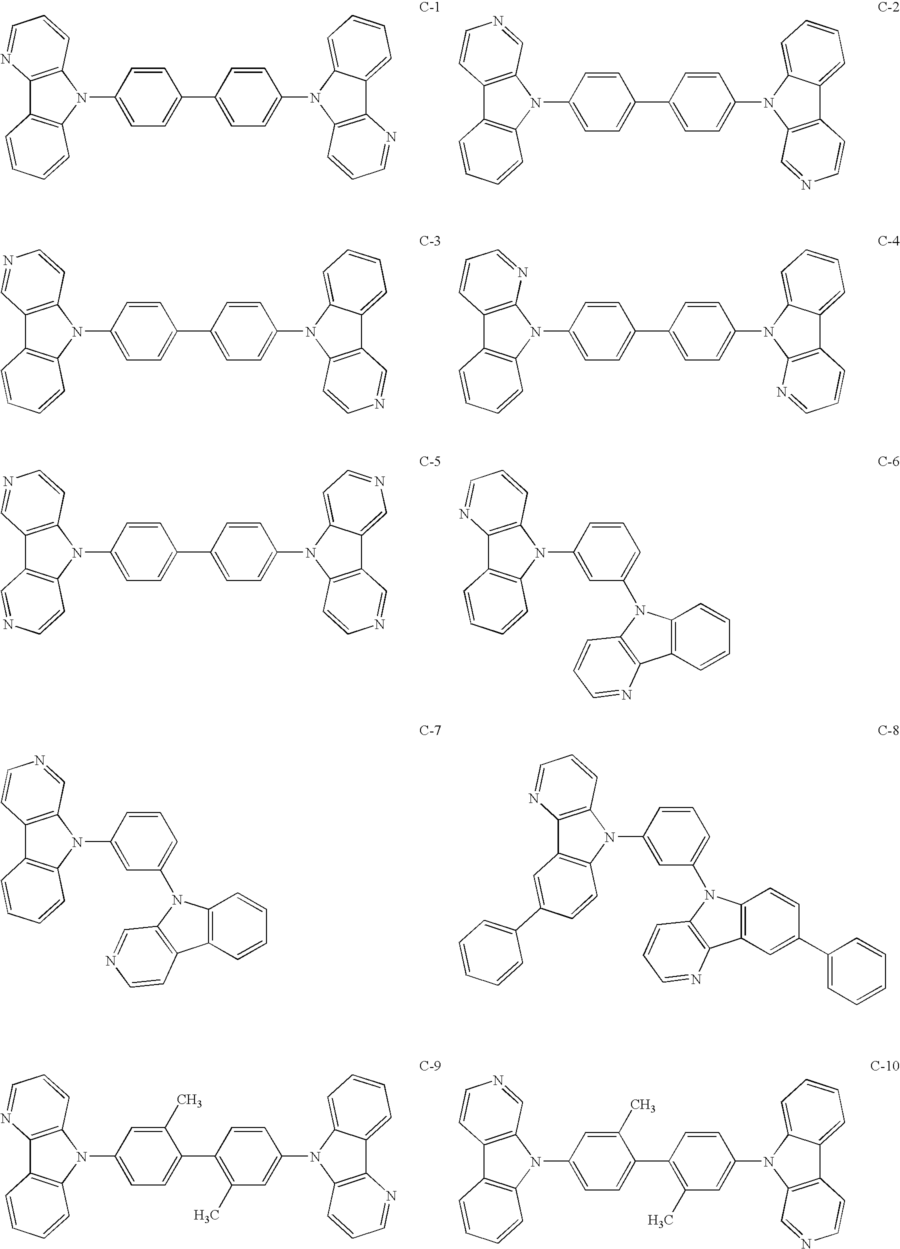

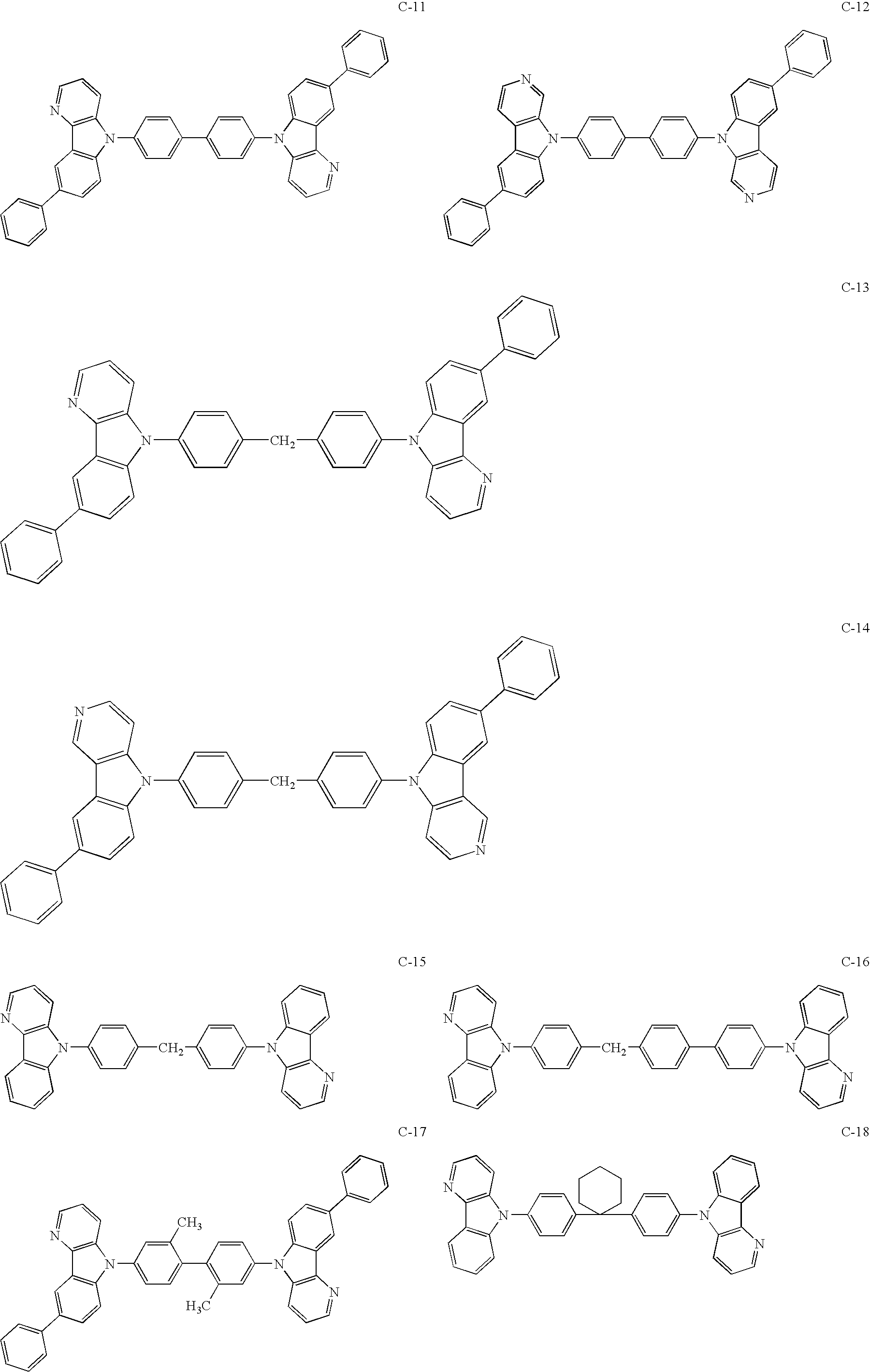

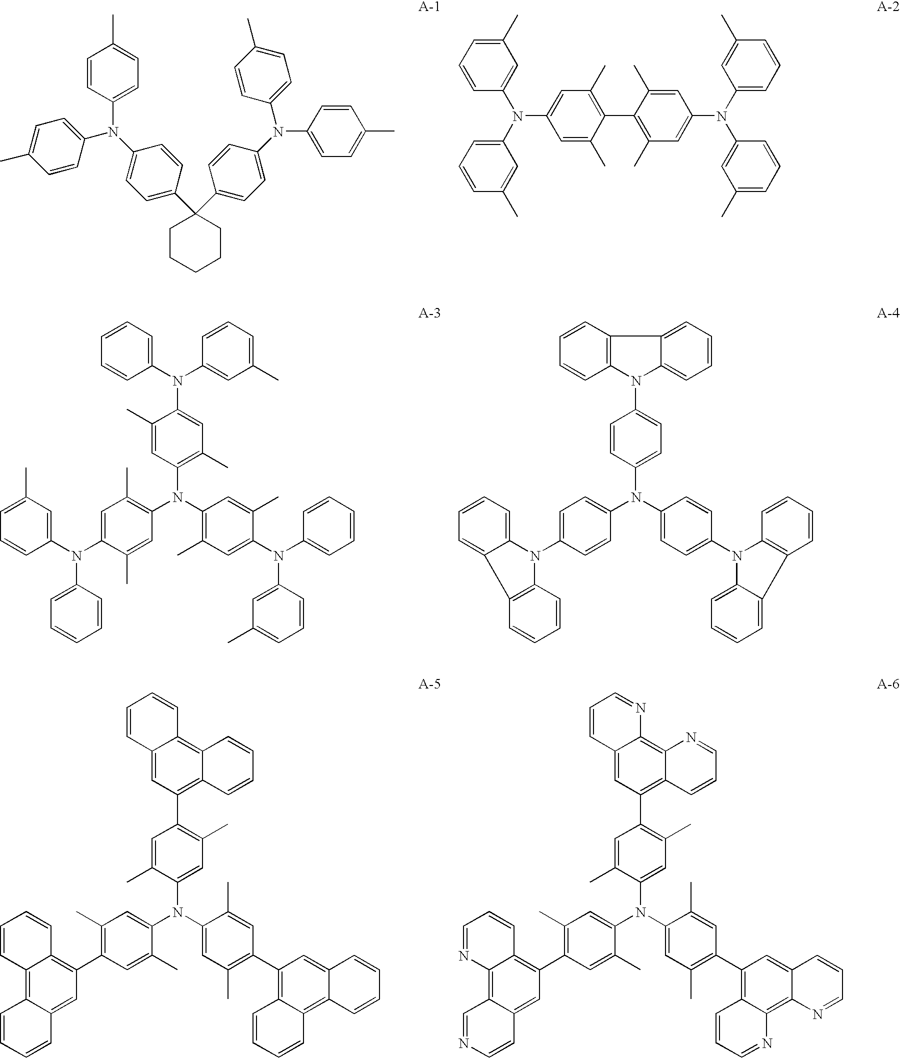

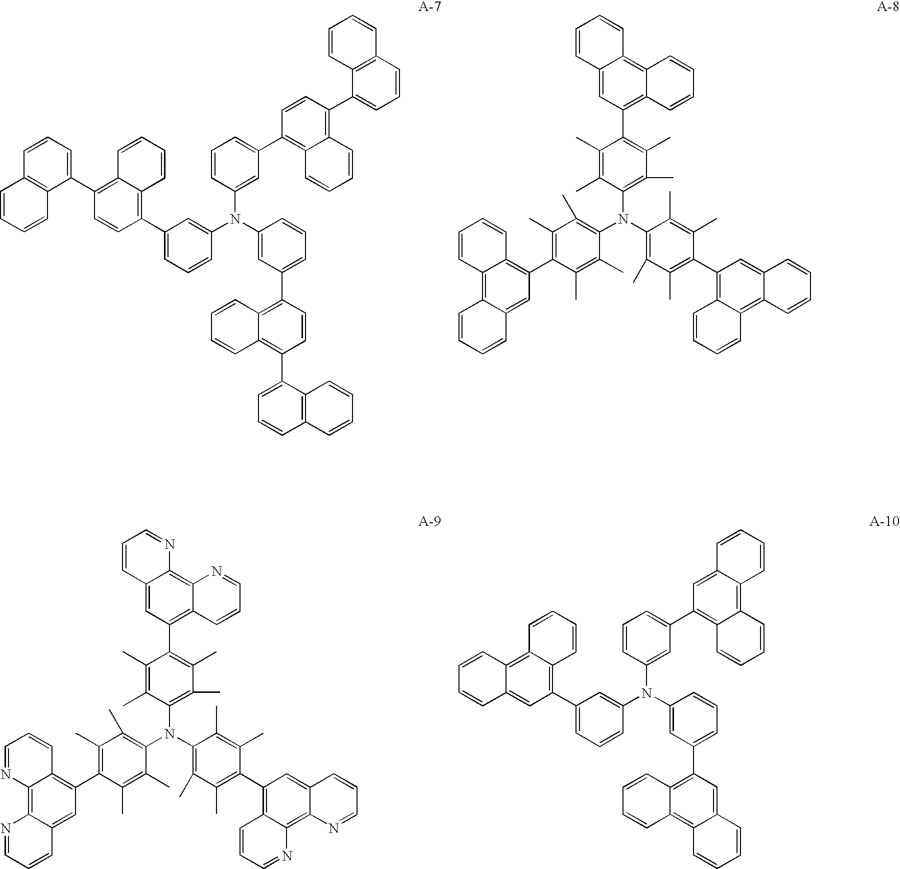

In the present invention, compounds represented by the following Formulae (1)-(6) are preferably provided as the dopant material. When these are employed, longer operating life can be achieved.

A compound having a partial structure represented by forgoing Formula (1) is first provided as the dopant material.

In foregoing Formula (1), X₁, X₂ and X₃ each represent a carbon or nitrogen atom; Z1 represents a residue to form a 5-member aromatic heterocycle; Z2 represents a 6-member aromatic ring, a 5-member aromatic heterocycle or a 6-member aromatic heterocycle; and M represents Ir or Pt.

Further, a compound having a partial structure represented by the following Formula (2) out of forgoing Formula (1) is preferable as the dopant material.

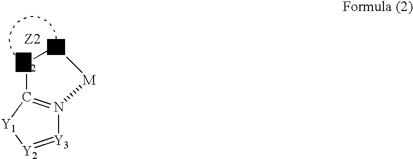

In Formula (2), X₂ and X₃ each represent a carbon or nitrogen atom; Y₁ represents NR₁, O or S, Y₂ and Y₂ each represent a carbon or nitrogen atom; Z2 represents a 6-member aromatic ring, a 5-member aromatic heterocycle or a 6-member aromatic heterocycle; M represents Ir or Pt; and R₁ represents a hydrogen atom, an aliphatic group, an aromatic group or a heterocyclic group.

Further, a compound having a partial structure represented by the following Formula (3) out of forgoing Formula (1) is preferable as the dopant material.

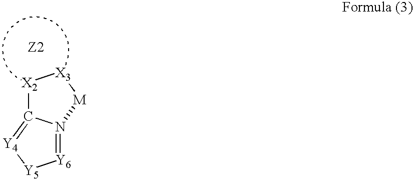

In Formula (3), X₂ and X₃ each represent a carbon or nitrogen atom; Y₅ represents NR₁, O or S; Y₄ and Y₆ each represent a carbon or nitrogen atom; Z2 represents a 6-member aromatic ring, a 5-member aromatic heterocycle or a 6-member aromatic heterocycle; M represents Ir or Pt; and R₁ represents a hydrogen atom, an aliphatic group, an aromatic group or a heterocyclic group.

Further, a compound having a partial structure represented by the following Formula (4) out of forgoing Formula (1) is preferable as the dopant material.

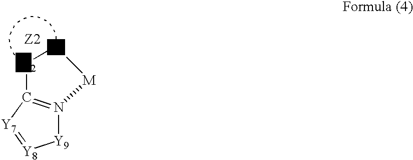

In Formula (4), X₂ and X₃ each represent a carbon or nitrogen atom; Y₉ represents NR₁, O or S; Y₇ and Y₈ each represent a carbon or nitrogen atom; Z2 represents a 6-member aromatic ring, a 5-member aromatic heterocycle or a 6-member aromatic heterocycle; M represents Ir or Pt; and R₁ represents a hydrogen atom, an aliphatic group, an aromatic group or a heterocyclic group.

Further, the dopant material having a partial structure represented by foregoing Formula (1) is preferably a compound having a partial structure represented by the following Formula (5).

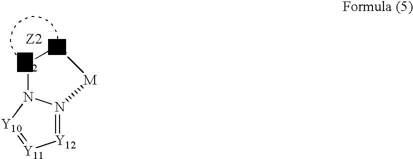

In Formula (5), X₂ and X₃ each represent a carbon or nitrogen atom; Y₁₀, Y₁₁ and Y₁₂ each represent a carbon or nitrogen atom; Z2 represents a 6-member aromatic ring, a 5-member aromatic heterocycle or a 6-member aromatic heterocycle; and M represents Ir or Pt.

In foregoing Formula (1), examples of the 5-member aromatic heterocycle include an oxazole ring, an oxadiazole ring, an oxatriazole ring, an isooxazole ring, a tetrazole ring, a thiadiazole ring, a thiatriazole ring, an isothiazole ring, a thiophene ring, a fran ring, a pyrrole ring, an imidazole ring, a pyrazole ring and so forth.

These rings each may possess substituents represented by R₂, R₃ or R₄ in after-mentioned Formula (6).

A benzene ring is provided as a 6-member aromatic cyclic hydrocarbon represented by Z2 in foregoing Formulae (1)-(6).

Further, examples of the 5-member aromatic heterocycle or 6-member aromatic heterocycle represented by Z2 in foregoing Formulae (1)-(6) include an oxazole ring, an oxadiazole ring, an oxatriazole ring, an isooxazole ring, a tetrazole ring, a thiadiazole ring, a thiatriazole ring, an isothiazole ring, a thiophene ring, a fran ring, a pyrrole ring, an imidazole ring, a pyrazole ring and so forth.

These rings each may possess substituents represented by R₂, R₃ or R₄ in after-mentioned Formula (6).

Further, examples of the aliphatic group represented by R₁ in foregoing Formulae (1)-(4) include a substituted or unsubstituted alkyl group such as a methyl group, an ethyl group, a propyl group, an iso-propyl group, a tert-butyl group, a pentyl group, a hexyl group, an octyl group, a dodecyl group, a tridecyl group, a tetradecyl group or a pentadecyl group; and a substituted or unsubstituted alkenyl group such as a vinyl group or an allyl group.

Further, examples of the aromatic ring group include a phenyl group, a nonylphenyl group, a naphthyl group and so forth.

Further, examples of the heterocyclic group include an aromatic heterocyclic group such as a furyl group, a thienyl group, a pyridyl group, a pyridazinyl group, a pyrimidinyl group, a pyridinyl group, a pyrazinyl group, a triazinyl group, an imidazolyl group, a pyrazolyl group, a thiazolyl group, a quinazolinyl group, or a phthalazinyl group; and a heterocyclic group such as a pyrrolidyl group, an imidazolidyl group, a morphoryl group or a oxazolidyl group.

These groups each may further possess a substituent. The after-mentioned groups are provided as the substituent.

In addition, in Formulae (1)-(5), it is preferable that Z2 is a residue to form a benzene ring.

Further, as the dopant material employed in the present invention, foregoing Formula (1) is preferably a compound having a partial structure represented by the following Formula (6).

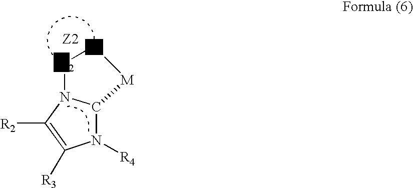

where X₂ and X₃ each represent a carbon or nitrogen atom; R₂, R₃ and R₄ each represent a hydrogen atom or a substituent; Z2 represents a 6-member aromatic ring; a 5-member aromatic heterocycle or a 6-member aromatic heterocycle; and M represents Ir or Pt.

Herein, the 6-member aromatic ring, and the 5-member or 6-member aromatic heterocycle represented by Z2 are the same as in the case of foregoing Formulae (1)-(5), and examples of substituents represented by R2, R3 and R4 include an alkyl group such as a methyl group, an ethyl group, a propyl group, an iso-propyl group, a tert-butyl group, a pentyl group, a hexyl group, an octyl group, a dodecyl group, a tridecyl group, a tetradecyl group or a pentadecyl group; a cycloalkyl group such as a cyclopentyl group or a cyclohexyl group; an alkenyl group such as a vinyl group or an allyl group; an alkynyl group such as a propalgyl group or the like; an aryl group (referred to also as an aromatic hydrocarbon group) such as a phenyl group, a tolyl group, a xylyl group, a naphthyl group, a biphenylyl group, an anthryl group or a phenanthryl group; a heterocyclic group such as a pyrrolidyl group, an imidazolidyl group, a morphoryl group or a oxazolidyl group; an aromatic heterocyclic group such as a pyridyl group, a pyridinyl group, a furyl group, pyrrolyl group, an imidazolyl group, a benzimidazolyl group, a pyrazolyl group, a pyrazinyl group, a triazolyl group (for example, a 1,2,4-triazolyl-1-yl group and 1,2,3-triazolyl-1-yl group), an oxazolyl group, a benzoxyazolyl group, a thiazolyl group, an iso-oxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, carbonylyl group, a diazacarbazolyl (a ring in which a carbon atom constituting a carboline ring is replaced by a nitrogen atom), a quinoxalinyl group, a pyridazinyl group, a triazinyl group, a quinazolinyl group or a phthalazinyl group; an alkoxyl group such as a methoxy group, an ethoxy group, a propyloxy group, a pentyloxy group, a hexyloxy group, an octyloxy group or dodecyloxy group; a cycloalkoxyl group such as cyclopentyloxy group and a cyclohexyloxy group; an aryloxy group such as a phenoxy group or a naphthyloxy group; an alkylthio group such as a methylthio group, an ethylthio group, a propylthio group, a pentylthio group, a hexylthio group, an octylthio group or a dodecylthio group; a cycloalkylthio group such as a cyclopentylthio group or a cyclohexylthio group; an arylthio group such as a phenylthio group or a naphthylthio group; an alkoxycarbonyl group such as a methyloxycarbonyl group, an ethyloxycarbonyl group, a butyloxycarbonyl group, an octyloxycarbonyl group or a dodecyloxycarbonyl group; an aryloxycarbonyl group such as a phenyloxycarbonyl group or a naphthyloxycarbonyl group; a sulfamoyl group such as an aminosulfonyl group, a methylaminosulfonyl group, a dimethylaminosulfonyl group, a butylaminosulfonyl group, a hexylaminosulfonyl group; a cyclohexylaminosulfonyl group, an octylaminosulfonyl group, a dodecylaminosulfonyl group, a phenylaminosulfonyl group, a naphthylaminosulfonyl group or a 2-pyridylaminosulfonyl group; a ureido group such as a methylureido group, an ethylureido group, a pentylureido group, a cyclohexylureido group, an octylureido group, a dodecylureido group, a phenylureido, a naphthylureido group or a 2-pyridylaminoureido group; an acyl group such as an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group or a pyridylcarbonyl group; an acyloxy group such as an acetyloxy group, an ethylcarbonyloxy group, a butylcarbonyloxy group, an octylcarbonyloxy group, a dodecylcarbonyloxy group or a phenylcarbonyloxy group; an amido group such as a methylcarbonylamino group, an ethylcarbonylamino group, a dimethylcarbonylamino group, a propylcarbonylamino group, a pentylcarbonylamino group, a cyclohexylcarbonylamino group, a 2-ethylhexylcarbonylamino group, an octylcarbonylamino group, a dodecylcarbonylamino group, a phenylcarbonylamino group or a naphthylcarbonylamino group; a carbamoyl group such as an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecylaminocarbonyl group, a phenylaminocarbonyl group, a naphthylaminocarbonyl group or a 2-pyridylaminocarbonyl group; a sulfinyl group such as a methylsulfinyl group, an ethylsulfinyl group, a butylsulfinyl group, a cyclohexylsulfinyl group, a 2-ethylhexylsulfinyl group, a dodecylsulfinyl group, a phenylsulfinyl group, a naphthylsulfinyl group or a 2-pyridylsulfinyl group; an alkylsulfonyl group or an arylsulfonyl group such as a methylsulfonyl group, an ethylsulfonyl group, a butylsulfonyl group, a cyclohexylsulfonyl group, a 2-ethylhexylsulfonyl group, a dodecylsulfonyl group, a phenylsulfonyl group, a naphthylsulfonyl group or a 2-pyridylsulfonyl group; an amino group such as an amino group, an ethylamino group, a dimethylamino group, a butylamino group, a cyclopentylamino group, a 2-ethylhexylamino group, a dodecylamino group, an anilino group, a naphthylamino group or a 2-pyridylamino group; a nitro group; and a cyano group.

Examples of the dopant material having partial structures represented by foregoing Formulae (1)-(6) are shown below, but the dopant material is not limited thereto.

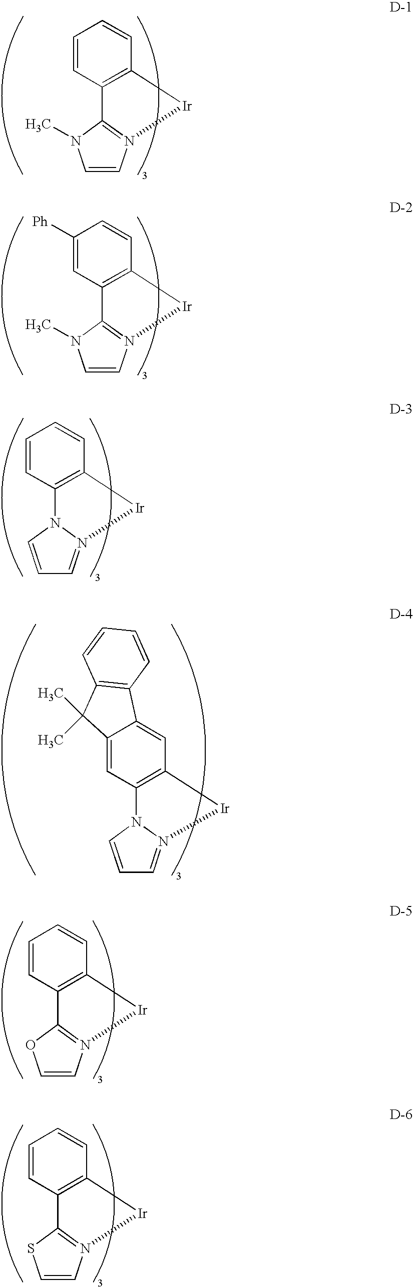

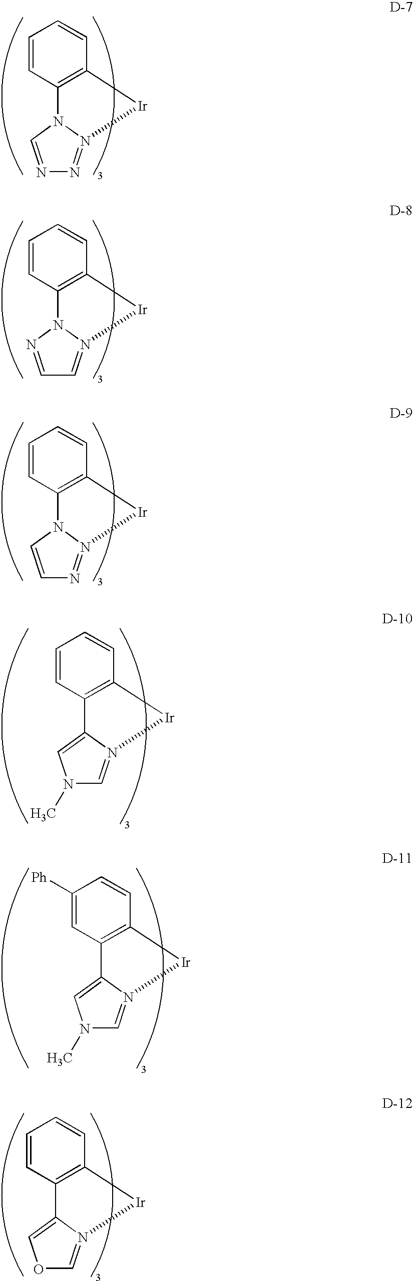

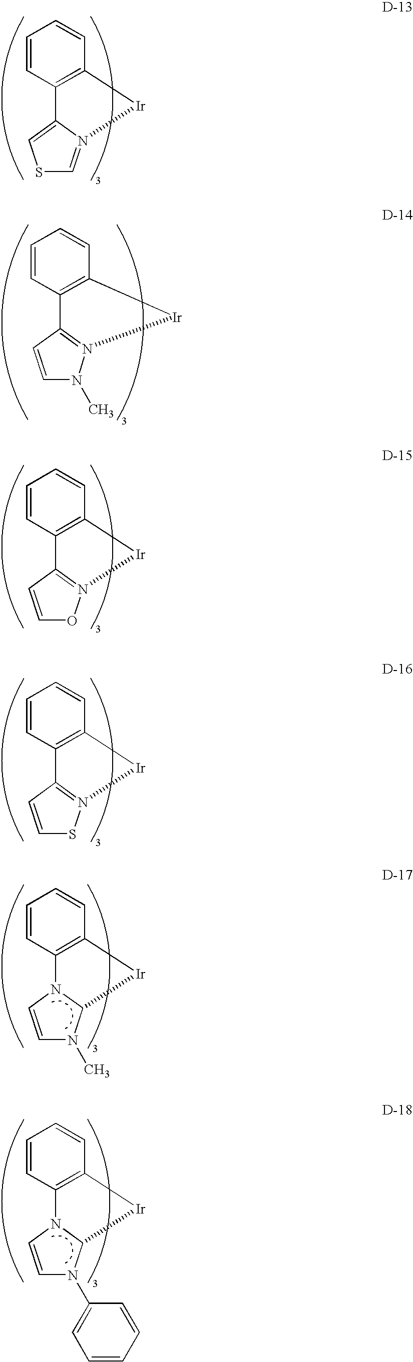

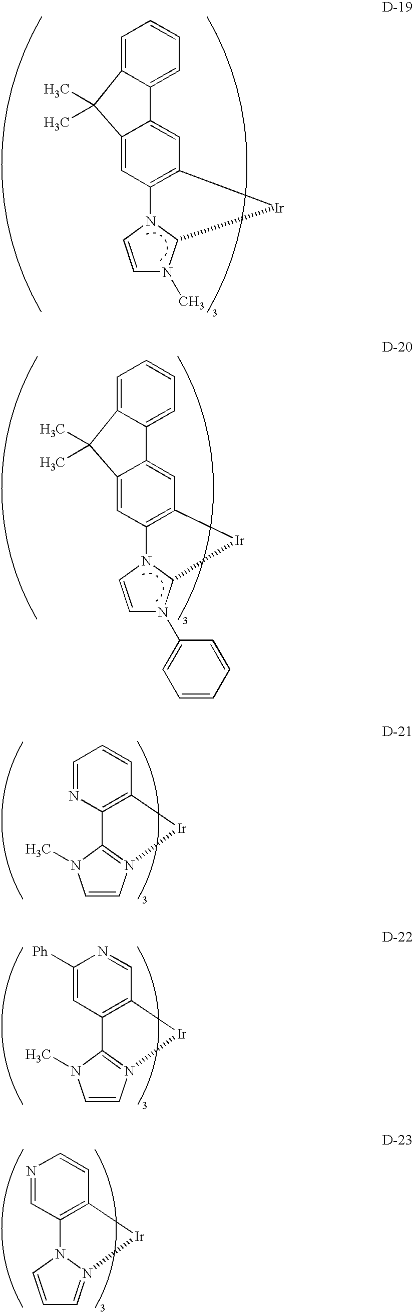

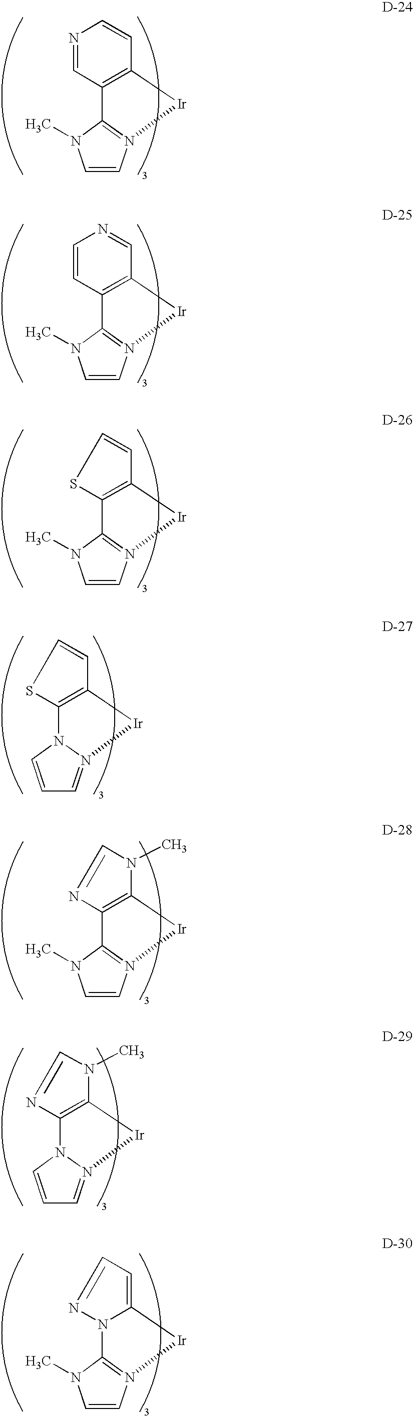

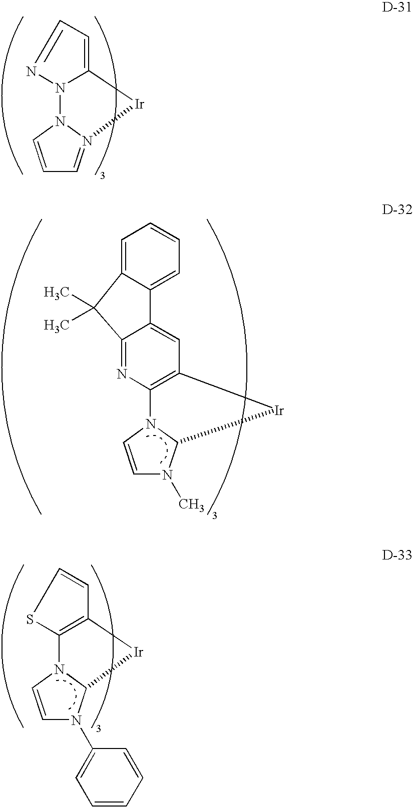

An intermediate layer of the present invention is referred to as a layer provided so as to be in contact on the anode side of an emission layer.

In the present invention, an intermediate material in the intermediate layer is an organic compound containing any one of carbazole, carboline and a triaryl amine structure. It is preferable that a compound exemplified as a host material for the foregoing emission layer is similarly employed, provided that the intermediate material is not limited thereto. If they are the foregoing host material, hole transport material and dopant material, as well as a material satisfying forgoing Formulae (2), (3), (5) or the like, those are preferably usable.

However, the intermediate material and the foregoing host material are preferably an identical compound.

A hole transport layer is composed of a hole transport material exhibiting the function of transporting holes, and in a broad sense, includes a hole injection layer and an electron blocking layer. The hole transport layer can be provided as a single layer or a plurality of layers.

Light emitted during returning from the excited singlet state to the ground state is called fluorescence, and light emitted during returning from the excited triplet state to the ground state is called phosphorescence. Since an internal quantum efficiency has an upper limit of 100% when an excited triplet is employed, the light emission efficiency quadruples in principle in comparison to the case of the excited singlet. Therefore, it is expected to be applied to a lighting device and so forth.

As to hole mobility, a mean traveling speed of holes in a thin film is proportionally increased with electric field, but a proportionality coefficient with respect to holes in this case is called hole mobility.

Similarly, as to electron mobility, a mean traveling speed of electrons in a thin film is proportionally increased with electric field, but a proportionality coefficient with respect to electrons in this case is called electron mobility.

The hole mobility and the electron mobility are measured via a Time-Of-Flight (T.O.F) method as described below, employing TOF-301 manufactured by OPTEL Co., Ltd., for example. A specimen obtained by sandwiching a thin film of a material which is measured between a semi-transparent electrode and a metal electrode is exposed to wave pulse, and the hole mobility and electron mobility can be determined via transient current characteristics of sheet-shaped carrier produced by the foregoing wave pulse.

Ionization potential is defined as energy to release electrons at HOMO (the highest occupied molecular orbital) of a compound to the vacuum level, and is specifically defined as energy to take out electrons from a compound in a film form (a layer form). The energy can directly be determined via photoelectron spectroscopy. These can be measured employing ESCA 5600 UPS (ultraviolet photoemission spectroscopy), produced by Ulvac-Phi, Inc., for example.

Further, triplet excitation energy of a dopant material, an intermediate material or the like can be calculated by measuring the 0-0 band of a phosphorescence spectrum of the material (compound).

Firstly, the 0-0 band of phosphorescence spectrum can be determined with a measuring method described below.

### <<Measuring Method of 0-0 Band of Phosphorescence Spectrum>>

A compound to be measured is dissolved in a mixed solvent of well-deoxygenated ethanol/methanol (4/1 by volume) and placed in a cell for phosphorescence measurement, followed by irradiation of exciting light at a liquid nitrogen temperature of 77 K to measure an emission spectrum 100 ms after completion of the irradiation of exciting light It is conceivable that since phosphorescence features a longer emission life than that of fluorescence, most of light remaining after 100 ms have elapsed is phosphorescence.

As to a compound insoluble in the solvent system described above, any solvent, which can dissolve the compound, may be employed (there is no substantial problem since a solvent effect on the phosphorescence wavelength in the above measuring method is negligible). In the present invention, the 0-0 band is defined as the maximum emission wavelength appearing on the shortest wavelength side in the phosphorescence spectrum chart obtained via the above measuring method.

Since intensity of a phosphorescence spectrum is generally weak, when the spectrum is magnified, it becomes difficult, in some cases, to distinguish between a noise band and a signal peak. In such the case, it is possible to determine a targeted signal peak in such a manner that a light emission spectrum generated right after irradiation of excitation light (for convenience, referred to as “stationary light spectrum”) is magnified, which is then superimposed on another magnified light emission spectrum generated at 100 ms after exposure to excitation light (for convenience, referred to as “phosphorescence spectrum”), to detect a peak wavelength from the stationary light spectrum originated in the phosphorescence spectrum.

The peak wavelength by separation of the noise band and the signal peak via a smoothing treatment is detected. The smoothing method by Savitzky and Golay is applied as the smoothing treatment.

### <<Emission Layer>>

The emission layer of the present invention is a layer, which emits light via recombination of electrons and holes injected from an electrode or a layer such as an electron transport layer or a hole transport layer. The emission layer may be composed of a plurality of emission layers each having a different light emission peak, or may be composed of a structure to form at least two kinds of emission colors by containing light-emitting materials each having a different light emission peak in a single layer. Further, in cases where a number of emission layers are more than 4, the layer having the identical light emission spectrum and maximum emission wavelength may be a plurality layers.

In order to prepare an emission layer, thin film formation of the foregoing emission dopant and a host compound can be conducted by a thin-film forming method well known in the art such as a vacuum evaporation method, a spin coating method, a cast method, an inkjet method and a LB method. The emission layer is preferably adjusted to have a thickness of 1-100 nm, and more preferably adjusted to have a thickness of 1-20 nm.

### (Host Compound)

The host compound contained in the emission layer in the organic EL element of the present invention is defined to be a compound in which phosphorescent emission has a phosphorescent quantum yield of less than 0.1 at room temperature (25° C.), and preferably has a phosphorescent quantum yield of less than 0.01 at room temperature (25° C.). Among compounds containing the emission layer, it is preferable to be a compound having a content of at least 20% by weight. It is possible to have a different luminescence mixture by employing phosphorescent compounds used as the emission dopant.

The host material of the present invention is preferably a compound containing any one of a carbazole ring, a carboline ring and a triaryl amine structure described before. The foregoing compound is preferably usable for the foregoing intermediate layer.

Further, an emission host used in the present invention may be a commonly known molecular weight compound, a polymeric compound having a repeating unit in the molecule, or a low molecular weight compound having a polymerizable group such as a vinyl group or an epoxy group (an evaporation polymerizable emission host). The commonly known host compound is preferably a compound with high Tg (glass transition temperature), which has a hole transporting capability and an electron transporting capability, and prevents the emission wavelength from shifting to longer wavelength. Specific examples of the commonly known host compound include compounds include those described in the following patent documents.

Examples thereof include Japanese Patent O.P.I. Publication No. 2001-257076, Japanese Patent O.P.I. Publication No. 2002-308855, Japanese Patent O.P.I Publication No. 2001-313179, Japanese Patent O.P.I. Publication No. 2002-319491, Japanese Patent O.P.I. Publication No. 2001-357977, Japanese Patent O.P.I. Publication No. 2002-334786, Japanese Patent O.P.I. Publication No. 2002-8860, Japanese Patent O.P.I. Publication No. 2002-334787, Japanese Patent O.P.I. Publication No. 2002-15871, Japanese Patent O.P.I. Publication No. 2002-105445, Japanese Patent O.P.I. Publication No. 2002-343568, Japanese Patent O.P.I. Publication No. 2002-141173, Japanese Patent O.P.I. Publication No. 2002-352957, Japanese Patent O.P.I. Publication No. 2002-203683, Japanese Patent O.P.I. Publication No. 2002-363227, Japanese Patent O.P.I. Publication No. 2002-231453, Japanese Patent O.P.I. Publication No. 2003-3165, Japanese Patent O.P.I. Publication No. 2002-234888, Japanese Patent O.P.I. Publication No. 2003-27048, Japanese Patent O.P.I. Publication No. 2002-255934, Japanese Patent O.P.I. Publication No. 2002-260861, Japanese Patent O.P.I. Publication No. 2002-280183, Japanese Patent O.P.I. Publication No. 2002-299060, Japanese Patent O.P.I. Publication No. 2002-302516, Japanese Patent O.P.I. Publication No. 2002-305033, Japanese Patent O.P.I. Publication No. 2002-305084, and Japanese Patent O.P.I. Publication No. 2002-308837.

In the present invention, not less than 50% by weight of the host compound has a phosphorescence emission energy of at least 2.9 eV and compounds each having a Tg (glass transition point) of at least 90° C. are preferable, and compounds each having a Tg (glass transition point) of at least 100° C. are further preferable.

### {Tg (Glass Transition Point)}

Herein, glass transition point Tg is a value obtained by a method in accordance with JIS-K-7121 employing DSC (Differential Scanning Calorimetry).

In order to obtain an organic EL element exhibiting emission efficiency, it is preferable in the present invention that not only a host compound is contained in an emission layer employed in the organic EL element of the present invention, but also a phosphorescence emission dopant is contained a dopant material.

However, in the present invention, a fluorescence emitter (also called a fluorescent dopant) can be contained in an emission layer as a dopant material.

Typical examples of the fluorescence emitter (fluorescent dopant) include a coumarine based dye, a pyrane based die, a cyanine based dye, a chloconium based dye, a squalenium based dye, an oxobenzanthracene based dye, a fluorescene based dye, a rhodamine based dye, a pyrylium based dye, a perylene based dye, a stilbene based dye, and a polythiophene based dye, and a rare earth element complex based phosphor. Further, a commonly known dopant is also usable.

### <<Intermediate Layer>>

In the present invention, a non-light emitting intermediate layer can be provided in the emission layer. The non-light emitting intermediate layer is one provided between emission layers of an emission layer unit in cases where there are a plurality of emission layers. The non-light emitting intermediate layer preferably has a thickness of 1-50 nm, but more preferably has a thickness of 3-10 nm to suppress the mutual interaction, such as an energy transfer, between the adjacent emission layers, and to result in no high load to electric current and voltage characteristics of elements.

Materials employed in the above non-light emitting intermediate layer may be the same as the host compounds of the emission layer or may differ, but it is preferable that they are the same as the host materials of at least one of two adjacent emission layers.

### <<Injection Layer: Electron Injection Layer, Hole Injection Layer>>

The injection layer provided according to necessity is classified into an electron injection layer and a hole injection layer. The injection layer may be provided between an anode and an emission layer or a hole transport layer, or between a cathode and an emission layer or an electron transport layer as described above. The injection layer is a layer provided between an electrode and an organic layer to improve a driving voltage drop and emission luminance. The injection layer is described in detail in “Yuuki EL soshi to sono kougyouka saizennsenn (Organic EL elements and forefront of their industrialization) Vol. 2, Sect. 2, “Electrode materials” pages 123-166, Nov. 30, 1998, published by NTS Inc., and includes the hole injection layer (anode buffer layer) and the electron injection layer (cathode buffer layer).

The anode buffer layer (hole injection layer) is described in detail in Japanese Patent O.P.I. Publication No 9-45479, Japanese Patent O.P.I. Publication No. 9-260062 and Japanese Patent O.P.I. Publication No. 8-288069, and specific examples thereof include a phthalocyanine buffer layer typically made of copper phthalocyanine, an oxide buffer layer typically made of vanadium oxide, an amorphous carbon buffer layer and a polymer buffer layer employing a conductive polymer such as polyaniline (emeraldine) or polythiophene.

The cathode buffer layer (electron injection layer) is also described in detail in Japanese Patent O.P.I. Publication No. 6-325871, Japanese Patent O.P.I. Publication No. 9-17574, Japanese Patent O.P.I. Publication No. 10-74586, and so forth, examples thereof include a metal buffer layer typically made of strontium or aluminum, an alkali metal compound buffer layer typically made of lithium fluoride, an alkaline earth metal compound buffer layer typically made of magnesium fluoride and an oxide buffer layer typically made of aluminum oxide. The buffer layer (injection layer) is preferably a very thin layer and the thickness thereof is preferably 0.1 nm-5 μm, depending on the material.

### <<Blocking Layer: Hole Blocking Layer and Electron Blocking Layer>>

Blocking layers other than basic layers of structure as thin organic compound layers are provided, if desired. Examples thereof include hole blocking layers described in Japanese Patent O.P.I. Publication No. 11-204258 and Japanese Patent O.P.I. Publication No. 11-204359, as well as on page 237 of “Yuki EL Soshi to Sono Kogyoka Saizensen (Organic EL elements and forefront of their industrialization)” (published by NTS Inc.; 30 November, 1998).

The hole blocking layer exhibits the function of an electron transport layer in a broad sense, which is composed of a material exhibiting minimized hole transportability while exhibiting the function of electron transportability, and makes it possible to enhance recombination probability of electrons and holes by blocking holes while transporting electrons.

Further, the structure of the after-mentioned electron transport layer can be utilized as a hole blocking layer of the present invention, if desired. It is preferable that the hole blocking layer provided in the organic EL element of the present invention is arranged to be provided adjacent to an emission layer.

Further, in the presence of a plurality of emission layers having a plurality of different luminescent colors, it is preferable that the emission layer having a maximum emission wavelength of the shortest wavelength among all the emission layers is closest to an anode, but in such the case, it is preferable that a hole blocking layer is additionally provided between the shortest wavelength layer and the emission layer closest to the anode next to the shortest wavelength layer.

Further, at least 50% by weight of a compound contained in the hole blocking layer provided at that position preferably has an ionization potential higher than 0.2 eV, with respect to that of a host compound in the foregoing shortest wavelength emission layer.

On the other hand, the electron blocking layer exhibits the function of a hole transport layer in a broad sense, which is composed of a material exhibiting minimized electron transportability while exhibiting the function of hole transportability, and makes it possible to enhance recombination probability of electrons and holes by blocking electrons while transporting holes.

Further, the structure of the after-mentioned hole transport layer can be utilized as an electron blocking layer of the present invention, if desired. The hole blocking layer and the electron transport layer of the present invention each preferably have a thickness of 3-100 nm, and more preferably have a thickness of 5-30 nm.

### <<Hole Transport Layer>>

The hole transport layer is composed of a hole transport material exhibiting the function of transporting holes, and in a broad sense, includes a hole injection layer and an electron blocking layer. The hole transport layer can be provided as a single layer or a plurality of layers. Hole transport materials are those which exhibit either injection or transportation of holes, or blocking of electrons, and may be either organic or inorganic compounds. Examples thereof include triazole derivatives, oxazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, and aniline based copolymers, as well as electrically conductive macromolecular oligomers, especially thiophene oligomers.

It is possible to employ the above materials as hole transport materials. In addition, it is preferable to employ porphyrin compounds, aromatic tertiary amine compounds, and styrylamine compounds. Of these, it is particularly preferred to employ aromatic tertiary amine compounds. Typical examples of the aromatic tertiary amine compounds and styrylamine compounds include N,N,N′,N′-tetraphenyl-4,4′-diaminophenyl, N,N′-diphenyl-N,N′-bis(3-methylphenyl)-[1,1′-biphenyl]-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl; 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4-diaminodiphenyl ether, 4,4′-bis(diphenylamino)quaterphenyl, N,N,N-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbene, and N-phenylcarbazole. Further listed are compounds having two condensed aromatic rings in the molecule such as 4,4′-bis[N-(1-naphthyl)-N-phenylamino]biphenyl (NPD), described in U.S. Pat. No. 5,061,569 and 4,4′,4′-tris[N-(3-methylphenyl)-N-phenylamino]triphenylamine (MTDATA) in which three triphenylamine units are linked in a starburst type, described in Japanese Patent O.P.I. Publication No. 4-308688. In addition, it is possible to employ polymer materials which are formed by introducing the above materials into polymer chains or in which the above materials are employed as a main chain.

Further, it is possible to employ inorganic compounds such as p type-Si or p type SiC as a hole injection material and a hole transport material. Utilized can be p type Hole transport materials described in Japanese Patent O.P.I. Publication No. 11-251067, and J. Huang et al. literature (Applied Physics Letters 80 (2002).

In the present invention, these materials are preferably used since light-emitting elements exhibiting higher efficiency can be obtained. It is possible to form a hole transport layer in such a way that the above hole transport materials are subjected to thin film formation with methods, known in the art, such as a vacuum evaporation method, a spin coating method, a casting method, a printing method including an ink-jet method, or an LB method.

The thickness of the hole transport layer is not particularly limited. The above thickness is commonly 5 nm-5 μm, but is preferably 5-200 nm. The above hole transport layer may have a single layer structure composed of at least one kind of the above materials. Hole transport materials exhibiting a high p type property, which have been doped with impurities, can also be utilized.

Examples thereof include those described in Japanese Patent O.P.I. Publication No. 4-297076, Japanese Patent O.P.I. Publication No. 2000-196140, and Japanese Patent O.P.I. Publication No. 2001-102175, as well as J. Appl. Phys., 95, 5773 (2004). In the present invention, it is preferable to utilize such the hole transport layer exhibiting a high p type property since elements in low power consumption are possible to be prepared.

### <<Electron Transport Layer>>

The electron transport layer is composed of a material exhibiting the function to transport electrons, and includes, in a broad sense, an electron injection layer and a hole blocking layer. The electron transport layer may be provided as a single layer or a plurality of layers. Heretofore, in the case of a single electron transport layer or a plurality of electron transport layers, electron transport materials (which also work as hole blocking materials), which are employed in the electron transport layer adjacent to the cathode electrode side with respect to the emission layer, have been applicable when they exhibit the function to transfer electrons injected from the cathode to the emission layer. As such materials, it is possible to employ any of those selected from the compounds known in the art. Examples thereof include nitro-substituted fluorene derivatives, diphenylquinone derivatives, thiopyran dioxide derivatives, carbodiimide, fluorenylydenemethane derivatives, anthraquinodimethane and anthrone derivatives, as well as oxadiazole derivatives.

Further employed as electron transport materials may be thiadiazole derivatives, which are prepared by replacing the oxygen atom of the oxadiazole ring in the above oxadiazole derivatives with a sulfur atom, as well as quinoxaline derivatives known as an electron attractive group.

Further, it is possible to employ polymer materials which are prepared by introducing any of the above materials into the polymer chain or in which any of the above materials are employed as the main chain of the polymer. Further employed as the electron transport materials may be metal complexes of 8-quinolinol derivatives such as tris (8-quinolinol) aluminum (Alq), tris (5,7-dichloro-8-quinolinol) aluminum, tris (5,7-dibromo-8-quinilinol) aluminum, tris (2-methyl-8-quinolinol) aluminum, tris (5-methyl-8-quinilinol) aluminum, or bis(8-quinolinol) zinc (Znq), or metal complexes in which the mail metal of these metal complexes is replaced by In, Mg, Cu, Ca, Sn, Ga, or Pb.

Other than these, preferably employed as the electron transport materials may be metal free or metal phthalocyanines, or compounds in which the end of the above phthalocyanine is substituted with an alkyl group or a sulfonic acid group.

Further employed as electron transport materials may be distyrylpyrazine derivatives, Still further employed as electron transport materials may be inorganic semiconductors such as n type-Si or n type-SiC in the same manner as in the hole injection layer and hole transport layer.

It is possible to form an electron transport layer in such a way that the above electron transport materials are subjected to thin film formation with methods, known in the art, such as a vacuum evaporation method, a spin coating method, a casting method, a printing method including an ink-jet method, or an LB method.

The thickness of the electron transport layer is not particularly limited. The above thickness is commonly 5 nm-5 μm, but is preferably 5-200 nm. The above electron transport layer may have a single layer structure composed of at least one kind of the above materials.

Electron transport materials exhibiting a high n type property, which have been doped with impurities, can be utilized. Examples thereof include those described in Japanese Patent O.P.I. Publication No. 4-297076, Japanese Patent O.P.I. Publication No. 10-270172, Japanese Patent O.P.I. Publication No. 2000-196140, and Japanese Patent O.P.I. Publication No. 2001-102175, as well as J. Appl. Phys., 95, 5773 (2004).

In the present invention, it is preferable to utilize such the electron transport layer exhibiting a high n type property since elements in low power consumption are possible to be prepared.

### <<Supporting Substrate>>

Supporting substrates (hereinafter, referred to also as substrates, base boards, base materials, or supports) are not particularly limited to kinds of glass and plastic, and may further be transparent or opaque. When light is output from the substrate side, the substrate is preferably transparent. It is possible to list, as preferably employed transparent substrates, glass, quartz, or a transparent resin film. Of these, the particularly preferred substrate is a resin film capable of exhibiting flexibility in the organic EL element.

Examples of the resin film material include polyester such as polyethylene terephthalate (PET), or polyethylene naphthalate (PEN); cellulose esters or derivatives thereof such as polyethylene, polypropylene, cellophane, cellulose diacetate, cellulose triacetate, cellulose acetate butyrate, cellulose acetate propionate (CAP), cellulose acetate phthalate (TAC), or cellulose nitrate; polyvinylidene chloride, polyvinyl alcohol, polyethylene vinyl alcohol, syndiotactic polystyrene, polycarbonate, norbornene resins, polymethylpentane, polyether ketone, polyimide, polyether sulfone (PES), polyphenylene sulfide, polysulfones, polyetherimide, polyetherketoneimide, polyamide, fluororesins, nylon, polymethyl methacrylate, acryl or polyacrylates, and cycloolefin based resins such as ARTON (a registered trade, produced by JSR Corp.) or APERU (a registered trade name, produced by Mitsui Chemical Co., Ltd.).

The surface of a resin film is formed from a film made of an inorganic compound or an organic compound, or from a hybrid film made of both of them. The resulting film is preferably a barrier film having a water vapor permeability (at 25° C. and 90% RH) of at most 0.01 g/(m²·24 hours), which is determined by the method based JIS K 7129-1987. Further, the above film is preferably a high barrier film having an oxygen permeability of at most 10⁻³ ml/(m²·24 hours·atm) and determined by the method based on JIS K 7126-1992 and a water vapor permeability of at most 10⁻³ g/(m²·24 hours). Furthermore, the above film is preferably a barrier film having a water vapor permeability of at most 10⁻⁵ g/(m²·24 hours) and an oxygen permeability of at most 10⁻⁵ ml/(m²·24 hours·atm).

As a material to prepare the barrier film formed on the resin film surface so as to make a high barrier film, employed may be those which exhibit the function to retard penetration of materials such as moisture or oxygen which degrade elements, and it is possible to employ, for example, silicon oxide, silicon dioxide, or silicon nitride.

Further, in order to decrease brittleness, it is preferable to form a laminated layer structure composed of an inorganic layer and a layer made of organic materials. The lamination order of the inorganic and organic layers is not particularly limited. It is preferable that both are alternately laminated several times.

### <<Sealing>>

As a sealing means employed for sealing of an organic EL element of the present invention, provided can be a method by which a sealing member adheres to an electrode and a substrate employing an adhesive, for example. The sealing member may be arranged to cover the display region of the organic EL element, and may be either in the form of an intaglio plate or a flat plate. Further, transparency and electric insulation are not particularly limited. Specifically listed are a glass plate, a polymer plate or film, and a metal plate or film. Examples of the glass plate may include specifically soda-lime glass, barium and strontium containing glass, lead glass, aluminosilicic acid glass, borosilicic acid glass, barium borosilicic acid glass, and quartz.

Further, examples of the polymer plate may include those composed of polycarbonate, acryl, polyethylene terephthalate, polyether sulfide or polysulfone, while listed as the metal plates may be those composed of at least one metal selected from the group consisting of stainless steel, iron, copper, aluminum, magnesium, nickel, zinc, chromium, titanium, molybdenum, silicon, germanium and tantalum, or an alloy composed of at least two metals selected from the above group. In the present invention, polymer and metal films are preferably usable since an element can be formed as a thin film element.

Further, it is preferable that the polymer film has an oxygen permeability of at most 10⁻³ ml/(m²·24 hours·atm), and a water vapor permeability (at 25° C. and a relative humidity of 90% RH) of at most 10⁻⁵ g/(m²·24 hours). In order to concave a sealing member, sand blasting, chemical etching and so forth are employed.

It is possible to list, as an adhesive, photocurable and thermocurable type adhesives each having a reactive vinyl group of an acrylic acid based oligomer and a methacrylic acid based oligomer, and moisture curable type adhesives such as 2-cyanoacrylic acid ester and so forth. It is also possible to list a thermal and chemical curing type (two liquid mixture). It is further possible to list hot-melt type polyamide, polyester, and polyolefin. It is still further possible to list cationically curable type UV curable type epoxy resin adhesives. Since organic EL elements are occasionally degraded because of a thermal treatment, preferred are those which are adhesion-curable from room temperature to 80° C. Further, desiccants may be dispersed into the above adhesives. Adhesives may be coated onto the sealing portion by a commercial dispenser or may be printed so as to print in the same manner as screen printing.

Further, inorganic and organic material layers are formed in such a configuration that in the outside of an electrode on the side which interposes an organic layer and faces a substrate, the foregoing electrode and organic layer are covered in the form of being in contact with the substrate. The above-described inorganic and organic layer is preferably employed as the sealing film. In this case, any of the materials may be applied to the foregoing film as long as they exhibit the function to retard penetration of materials, such as moisture or oxygen, which result in degradation of the element. Usable examples thereof include silicon oxide, silicon dioxide, and silicon nitride. Further, in order to improve flexibility of the foregoing film, it is preferable that a laminated layer structure is realized by employing these inorganic layers and layers composed of organic materials.

### <<Protective Film and Protective Plate>>

In order to enhance mechanical strength of the element, a protective film or a protective plate may be provided on the outer side of the above sealing film on the side facing a substrate, while interposing an organic layer or the above sealing film. Specifically, when sealing is conducted via application of the above sealing film, the resulting strength is not always sufficient. Consequently, it is preferable to provide the above protective film or protective plate. It is possible to employ, as usable materials for the above, glass plates, polymer plate or polymer film, and metal plate or metal film which are the same as those employed for the above sealing. In view of lightweight and thin film formation, it is preferable to employ polymer films.

### <<Method of Preparing Organic EL Element>>

As an example of the method of preparing an organic EL element of the present invention, described is a method of preparing an organic EL element composed of anode/hole injection layer/hole transport layer/emission layer/hole blocking layer/electron transport layer/cathode. An anode is initially prepared in such a manner that a thin film composed of desired electrode materials, such as anode materials, is formed on an appropriate substrate so as to produce a film thickness of at most 1 μm, but preferably a film thickness of 10-200 nm via evaporation, sputtering or such. Subsequently, formed thereon is an organic compound thin film composed of a hole injection layer, a hole transport layer, an intermediate layer of the present invention, an emission layer, a hole blocking layer and an electron transport layer. Examples of the method of forming this organic compound thin film include, as described above, an evaporation method and wet processes (such as a spin coating method, a casting method, an inkjet method, and a printing methods but specifically preferred are a vacuum evaporation method, a spin coating method, an inkjet method and a printing method to conduct film formation in view of easy preparation of a homogeneous film and inhibition of pin hole formation.

Further, a different film forming method for each layer may be applied. When an evaporation method is employed for film formation, the evaporation condition depends on kinds of utilized compounds, but preferably usable are, in general, those such as a boat heating temperature of 50-450° C., a vacuum degree of 10⁻⁶-10⁻² Pa, an evaporation rate of 0.01-50 nm/sec, a substrate temperature of from −50° C. to 300° C., and a film thickness of 0.1 nm-5 μm. A film thickness of 5-200 nm is more preferable.

After forming these layers, a thin film composed of a cathode material is formed thereon with an evaporation method, a sputtering method or the like so as to give a film thickness of at most 1 μm, but preferably a film thickness of 50-200 nm to form a cathode, and a desired organic EL element is obtained.

As to preparation of this organic EL element, all the way through from a hole injection layer to a cathode may be prepared with just one vacuuming process, or a different film forming method to remove the specimen from the evaporator along the way may be conducted. In this case, the operation should be conducted under the drying and inert gas atmosphere condition.

Further, the above preparation order is possible to be reversed. When direct current voltage is applied to the resulting multicolor display device obtained in such a way, a voltage of approximately 2-40 V is applied while the anode is employed at positive polarity, and the anode is employed at negative polarity, whereby it is possible to observe luminescence. Further, an alternating current voltage may also be applied. In addition, waveform of the applied alternating current is not limited.

### <<Application>>

An organic electroluminescent element of the present invention is usable as display devices, displays, and various luminescent sources.

Examples of the lighting device utilized for an organic EL element of the present invention as the luminescent source include home lighting devices and lighting devices in vehicles, backlights for clocks and liquid crystals, advertising boards, traffic lights, light sources for optical memory media, light sources for electrophotographic copiers, light sources for optical communication processors, and light sources for optical sensors, but the present invention is hot limited thereto.

Further, an organic EL element may be used as a kind of lamp such as a lighting source or an exposure light source, and be also used as an image-projection type projector or a direct visibility type display device for still and moving images.

As a driving system to utilize for a display device for moving image reproduction, both a direct matrix (passive matrix) system and an active matrix system may be allowed to be employed.

Further, a full color display device is possible to be produced by using at least three kinds of organic EL elements of the present invention each having different luminescent color.

Or, each of B, G and R light is possible to be removed from homochromatic luminescent color, for example, white luminescence employing a color filter to produce full color.

Further, luminescent color of an organic EL element is also possible to be converted into another color employing a color conversion filter to produce full color, but in this case, organic EL emission having a λmax of at most 480 nm is preferable.

### EXAMPLE

Next, the present invention will be explained referring to examples, but the present invention is not limited thereto.

### Example 1

A substrate (NA45, produced by NH Techno Glass Corp.), prepared by forming a 100 nm thick ITO (indium tin oxide) film as an anode on a glass plate having a size of 100×100×1.1 mm, was subjected to patterning, and a transparent supporting substrate provided with this ITO transparent electrode was cleaned with isopropyl alcohol via ultrasonic waves, followed by being dried employing dray nitrogen gas and being cleaned for 5 minutes employing UV ozone.

This transparent supporting substrate was fixed on a substrate holder in a commercially available vacuum evaporator, and installed in the vacuum evaporator. On the other hand, inside the vacuum evaporator, 200 mg of CuPc were charged in a molybdenum resistance heating boat; 200 mg of α-NPD were charged in another molybdenum resistance heating boat; 100 mg of m-MTDATXA were charged in another molybdenum resistance heating boat; 100 mg of D-1 were charged in another molybdenum resistance heating boat; 100 mg of HB-1 were charged in another molybdenum resistance heating boat; and 200 mg of BAlq were further charged in another molybdenum resistance heating boat.

Next, after depressurizing the vacuum chamber to 4×10⁻⁴ Pa, the foregoing heating boat in which CuPc was charged was heated via electricity application to form a hole injection layer having a thickness of 20 nm on a transparent supporting substrate via evaporation at an evaporation rate of 0.1 nm/sec. Further, the foregoing heating boat in which α-NPD was charged was heated via electricity application to form a hole transport layer having a thickness of 100 nm on the foregoing hole injection layer via evaporation at an evaporation rate of 0.1 nm/sec.

Next, the foregoing heating boat in which m-MTDATXA was charged and the foregoing heating boat in which D-1 was charged were heated via electricity application to form an emission layer having a thickness of 40 nm on the foregoing hole transport layer via co-evaporation at an evaporation rate of 0.1 nm/sec and at an evaporation rate of 0.06 nm/sec, respectively. Further, the foregoing heating boat in which HB-1 was charged was heated via electricity application to form a hole blocking layer on the foregoing emission layer.

Next, the foregoing heating boat in which BAlq was charged was heated via electricity application to form an electron transfer layer having a thickness of 40 nm on the foregoing hole blocking layer via evaporation at an evaporation rate of 0.1 nm/sec. In addition, the substrate temperature during evaporation was room temperature. Subsequently, 0.5 nm thick lithium fluoride was prepared via evaporation as a cathode buffer layer, and 110 nm thick aluminum was further prepared via evaporation for a cathode to produce organic EL element 1-1.

Organic EL elements 1-2-1-4 were prepared similarly to preparation of organic EL elements 1-1, except that one in which 10 nm thick m-MTDATXA was inserted between an emission layer and a hole transport layer as an intermediate layer in organic EL element 1-1 was arranged to be set to organic E1 element 1-2; one in which a host material of an emission layer was designed to be H-A in organic EL element 1-1 was arranged to be set to organic EL element 1-3; and one in which 10 nm thick H-A was inserted between an emission layer and a hole transport layer as an intermediate layer in organic EL element 1-3 was arranged to be set to organic EL element 1-4.

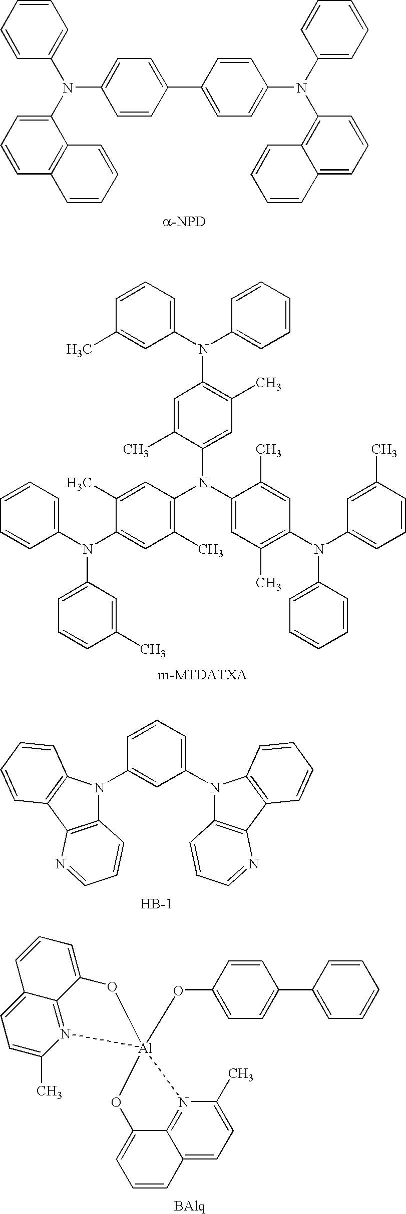

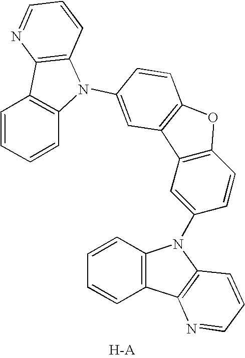

The following ionization potential of each of the host material, the intermediate material and the dopant material was measured via photoelectron spectroscopy employing ESCA 5600 UPS manufactured by ULVAC-PHI Co., Ltd.

| | |
|---|---|
| Ionization potential of | E1 = 5.5 eV |
| hole transport material (α-NPD) | |
| (Ionization potential of the intermediate | E2 = E3 = 5.5 eV |
| material or the host material) | |
| Ionization potential of m-MTDATXA | |
| Ionization potential of H-A | E2 = E3 = 6.1 eV |
| Ionization potential of dopant material (D-1) | E4 = 5.2 eV |

These ionization potentials satisfy foregoing Formulae (2) and (3).

Further, H-A is an electron transporting host material, which is namely a material satisfying a relationship of μe>μh, wherein μe represents electron mobility, and μh represents hole mobility. Those μh and μe were measured via a Time-Of-Flight (T.O.F) method employing TOF-301 manufactured by OPTEL Co., Ltd.

In contrast, m-MTDATXA is an electron transporting host material, which is namely a material satisfying a relationship of μe<μh, wherein μe represents electron mobility, and μh represents hole mobility. Those μh and μe were similarly measured employing TOF-301 manufactured by OPTEL Co., Ltd.

### <<Evaluation of Organic EL Elements 1-1-1-4>>

Organic EL elements 1-1-1-4 prepared as described in Example 1 were evaluated, and results thereof are shown in Table 1.

As to emission lifetime of each element shown in Table 1, each organic EL element prepared as described before was driven with driving voltage (V) to produce a front luminance of 1000 cd/m² to take time until reaching half decay time in luminance, and the values were expressed by relative values when element 101 value was set to 100%. In order to measure the front luminance, front luminance at a viewing field angle of 2° was measured in a visible light wavelength range between 430 and 480 nm in such a way that the optical axis of the spectroradiometric luminance meter is identical to the normal line from the light emission plane, employing a spectroradiometric luminance meter CS-1000 manufactured by Konica Minolta Sensing, Inc. to obtain integral intensity thereof.

In addition, organic EL element 1-1 was compared with organic EL element 1-2, and organic EL element 1-3 was compared with organic EL element 1-4. The values were expressed by relative values when each value of organic EL element 1-1 and organic EL element 1-3 was set to 100%.

TABLE 1

| | | Inter- | Hole mobility (μh) and | Lifetime in | |
|---|---|---|---|---|---|
| | Host | mediate | electron mobility (μe) | relative | |
| Element | material | layer | of host material | value (%) | |
| 1-1 | m-MTDATXA | Not | μe < μh | 100 | Comparative |
| | | provided | | | example |
| 1-2 | m-MTDATXA | m-MTDATXA | μe < μh | 105 | Present |
| | | | | | invention |
| 1-3 | H-A | Not | μe > μh | 100 | Comparative |
| | | provided | | | example |
| 1-4 | H-A | H-A | μe > μh | 110 | Present |
| | | | | | invention |

As is clear from Table 1, it is to be understood that the organic EL element of the present invention possessing an intermediate layer, which satisfies the relationship of forgoing Formulae (2) and (3) concerning each inter-ionization potential exhibits longer lifetime, and at this time, the longer lifetime is effectively produced when the electron mobility of the host material is larger than the hole mobility.

### Example 2

A substrate (NA45, produced by NH Techno Glass Corp.), prepared by forming a 100 nm thick ITO (indium tin oxide) film as an anode on a glass plate having a size of 100×100×1.1 mm, was subjected to patterning, and a transparent supporting substrate provided with this ITO transparent electrode was cleaned with isopropyl alcohol Via ultrasonic waves, followed by being dried employing dry nitrogen gas and being cleaned for 5 minutes employing UV ozone.

This transparent supporting substrate was fixed on a substrate holder in a commercially available vacuum evaporator, and installed in the vacuum evaporator. On the other hand, inside the vacuum evaporator, 200 mg of CuPc were charged in a molybdenum resistance heating boat, 200 mg of α-NPD were charged in another molybdenum resistance heating boat; 300 mg of H-1 were charged in another molybdenum resistance heating boat; 100 mg of D-1 were charged in another molybdenum resistance heating boat; 200 mg of HE-1 were charged in another molybdenum resistance heating boat; and 200 mg of Alq₃ were further charged in another molybdenum resistance heating boat.

Next, after depressurizing the vacuum chamber to 4×10⁻⁴ Pa, the foregoing heating boat in which CuPc was charged was heated via electricity application to form a hole injection layer having a thickness of 20 nm on a transparent supporting substrate via evaporation at an evaporation rate of 0.1 nm/sec. Further, the foregoing heating boat in which α-NPD was charged was heated via electricity application to form a hole transport layer having a thickness of 50 nm on a transparent supporting substrate via evaporation at an evaporation rate of 0.1 nm/sec.

Next, the foregoing heating boat in which H-1 was charged was heated via electricity application to form an intermediate layer having a thickness of 10 nm via evaporation at an evaporation rate of 0.1 nm/sec.

Further, the foregoing heating boat in which H-1 was charged, and the foregoing heating boat in which D-1 was charged were heated via electricity application to form an emission layer having a thickness of 30 nm on the foregoing hole transport layer via co-evaporation at an evaporation rate of 0.2 nm/sec and at an evaporation rate of 0.01 nm/sec, respectively.

Further, the foregoing heating boat in which HB-1 was charged was heated via electricity application to form a hole blocking layer having a thickness of 10 nm on an emission layer via evaporation at an evaporation rate of 0.1 nm/sec. Further, the foregoing heating boat in which Alq₃ was charged was heated via electricity application to form an electron transport layer having a thickness of 40 nm on a hole blocking layer via evaporation at an evaporation rate of 0.1 nm/sec.

In addition, the substrate temperature during evaporation was room temperature. Subsequently, 0.5 nm thick lithium fluoride was prepared via evaporation as a cathode buffer layer, and 110 nm thick aluminum was further prepared via evaporation for a cathode to produce organic EL element 2-1.

Organic EL elements 2-2 and 2-3 were prepared similarly to preparation of organic EL element 2-1, except that in organic EL element 2-1, one in which the intermediate layer having a thickness of 10 nm was replaced by an intermediate layer having a thickness of 7 nm was arranged to be set to organic EL element 2-2, and for comparison, one in which no intermediate layer was provided was arranged to be set to organic EL element 2-3.

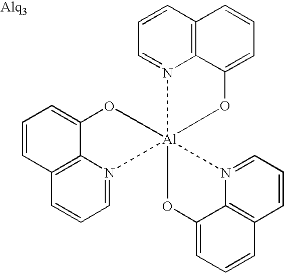

Ionization potential (E2, E3) of H-1 employed herein as an intermediate material was 6.2 eV, satisfying the relationship concerning a hole transport material, a host material and a dopant material, basically expressed by Formulae (2) and (3).

Ionization potential of D-1 employed as a dopant material was 5.3 eV, but triplet excitation energy calculated by measuring 0-0 band of the phosphorescence spectrum was at least 2.58 eV.

### <<Evaluation of Organic EL Elements 2-1-2-3>>

The resulting organic EL elements 2-1-2-3 were evaluated similarly to evaluation of Example 1, and results thereof are shown in Table 2.

In addition, organic EL element 2-1 and organic EL element 2-2 were compared with organic EL element 2-3, and measured results of emission lifetime in Table 2 were expressed by relative values when the emission lifetime of organic EL element 2-3 was set to 100%.

TABLE 2

| | | Inter- | Intermediate | Lifetime in | |
|---|---|---|---|---|---|
| | Host | mediate | layer | relative | |
| Element | material | layer | thickness | value (%) | |
| 2-1 | H-1 | H-1 | 10 nm | 300 | Present |
| | | | | | invention |
| 2-2 | H-1 | H-1 | 7 nm | 500 | Present |
| | | | | | invention |
| 2-3 | H-1 | Not | Not | 100 | Comparative |
| | | provided | provided | | example |

Herein, it is to be understood that an organic EL element of the present invention possessing an intermediate layer, which satisfies the formula in Structures 9, 22 and 35, exhibits improved lifetime, and the range indicated in the formula exhibits largely improved lifetime together with excellent light emission even in cases where thickness of the intermediate layer is varied.

### Example 3

A substrate (NA45, produced by NH Techno Glass Corp.), prepared by forming a 100 nm thick ITO (indium tin oxide) film as an anode on a glass plate having a size of 100×100×1.1 mm, was subjected to patterning, and a transparent supporting substrate provided with this ITO transparent electrode was cleaned with isopropyl alcohol via ultrasonic waves, followed by being dried employing dry nitrogen gas and being cleaned for 5 minutes employing UV ozone.

This transparent supporting substrate was fixed on a substrate holder in a commercially available vacuum evaporator, and installed in the vacuum evaporator. On the other hand, inside the vacuum evaporator, 200 mg of CuPc were charged in a molybdenum resistance heating boat; 200 mg of α-NPD were charged in another molybdenum resistance heating boat; 300 mg of H-1 were charged in another molybdenum resistance heating boat; 100 mg of D-1 were charged in another molybdenum resistance heating boat; 200 mg of HB-1 were charged in another molybdenum resistance heating boat; 200 mg of TNATA were charged in another molybdenum resistance heating boat; and 200 mg of Alq₃ were further charged in another molybdenum resistance heating boat.

Next, after depressurizing the vacuum chamber to 4×10⁻⁴ Pa, the foregoing heating boat in which CuPc was charged was heated via electricity application to form a hole injection layer having a thickness of 20 nm on a transparent supporting substrate via evaporation at an evaporation rate of 0.1 nm/sec. Further, the foregoing heating boat in which α-NPD was charged was heated via electricity application to form a hole transport layer having a thickness of 50 nm on a transparent supporting substrate via evaporation at an evaporation rate of 0.1 nm/sec.

Next, the foregoing heating boat in which TNATA was charged was heated via electricity application to form an intermediate layer having a thickness of 10 nm via evaporation at an evaporation rate of 0.1 nm/sec.

Further, the foregoing heating boat in which H-1 was charged, and the foregoing heating boat in which D-1 was charged were heated via electricity application to form an emission layer having a thickness of 30 nm on the foregoing hole transport layer via co-evaporation at an evaporation rate of 0.2 nm/sec and at an evaporation rate of 0.01 nm/sec, respectively.

Further, the foregoing heating boat in which HB-1 was charged was heated via electricity application to form a hole blocking layer having a thickness of 10 nm on an emission layer via evaporation at an evaporation rate of 0.1 nm/sec.

Further, the foregoing heating boat in which Alq₃ was charged was heated via electricity application to form an electron transport layer having a thickness of 40 nm on a hole blocking layer via evaporation at an evaporation rate of 0.1 nm/sec.

In addition, the substrate temperature during evaporation was room temperature. Subsequently, 0.5 nm thick lithium fluoride was prepared via evaporation as a cathode buffer layer, and 110 nm thick aluminum was further prepared via evaporation for a cathode to produce organic EL element 3-1.

Organic EL elements 3-2-3-5 were prepared similarly to preparation of organic EL element 3-1, except that in organic EL element 3-1, one in which intermediate material TNATA in the intermediate layer was replaced by H-20 was arranged to be set to organic EL element 3-2; one in which the intermediate material was replaced by m-MTDATXA was arranged to be set to organic EL element 3-4; one in which both the intermediate layer and the host material were replaced by m-MTDATXA was arranged to be set to organic EL element 3-S; and for comparison, one in which no intermediate layer was provided was arranged to be set to organic EL element 3-3.

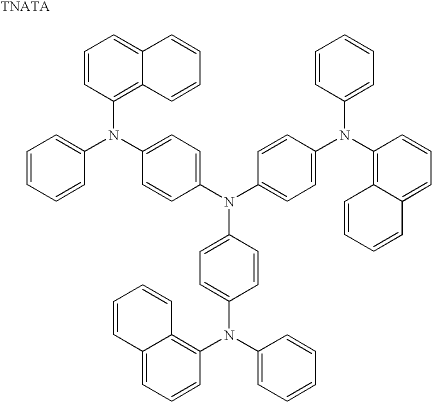

Concerning hole mobility of α-NPD as the hole transport material, hole mobility of each of TNATA and H-20 (L-394) as the intermediate material, and hole mobility of m-MTDATXA, the following relationships are allowed to be provided.

μ1 (α-NPD)>μ2 (TNATA)

μ1 (α-NPD)>μ2 (H-20)

μ1 (α-NPD)<μ2 (m-MTDATXA)

In addition, in order to determine the hole mobility, an evaporation layer having a thickness of 2000 nm was prepared on a glass substrate fitted with ITO to provide a metal electrode, and the system was exposed to light pulse from the glass side to measure each of transient current characteristics via a TOF method employing TOF-301 manufactured by OPTEL Co., Ltd., and to determine the magnitude relation based on α-NPD from carrier arrival time (t).

Further, when electron mobility μe and hole mobility μh of the host material were also checked, it was confirmed that not m-MTDATXA but host material H-1 satisfied μe>μh.

### <<Evaluation of Organic EL Elements 3-1-3-5>>

The resulting organic EL elements 3-1-3-5 were evaluated similarly to evaluation of Example 1, and results thereof are shown in Table 3.

Measured results of emission lifetime in Table 3 were expressed by relative values when the emission lifetime of organic EL element 3-3 was set to 100%.

TABLE 3

| | | | | | | Lifetime in | |
|---|---|---|---|---|---|---|---|
| | | | Host | | | relative | |
| *1 | *2 | *3 | material | *4 | *5 | value (%) | |
| 3-1 | α-NPD | TNATA | H-1 | μe > μh | *2 > *3 | 150 | Inv. |
| 3-2 | α-NPD | H-20 | H-1 | μe > μh | *2 > *3 | 250 | Inv. |
| 3-3 | α-NPD | Not | H-1 | μe > μh | — | 100 | Comp. |
| | | provided | | | | | |
| 3-4 | α-NPD | m-MTDATXA | H-1 | μe > μh | *2 < *3 | 110 | Inv. |
| 3-5 | α-NPD | m-MTDATXA | m-MTDATXA | μe < μh | *2 < *3 | 105 | Inv. |

*1: Element
*2: Hole transport material
*3: intermediate layer
*4: Hole mobility (μh) and electron mobility (μe) of host material
*5: Hole mobility of hole transport material and hole mobility of intermediate layer
Inv.: Present invention
Comp.: Comparative example

As is clear from Table 3, it is to be understood that organic EL elements of the present invention exhibit longer lifetime.

### Example 4

A substrate (NA45, produced by NH Techno Glass Corp.), prepared by forming a 100 nm thick ITO (indium tin oxide) film as an anode on a glass plate having a size of 100×100×1.1 mm, was subjected to patterning, and a transparent supporting substrate provided with this ITO transparent electrode was cleaned with isopropyl alcohol via ultrasonic waves, followed by being dried employing dry nitrogen gas and being cleaned for 5 minutes employing UV ozone. This transparent supporting substrate was fixed on a substrate holder in a commercially available vacuum evaporator, and installed in the vacuum evaporator on the other hand, inside the vacuum evaporator, 200 mg of CuPc were charged in a molybdenum resistance heating boat; 200 mg of α-NPD were charged in another molybdenum resistance heating boat; 300 mg of H-1 were charged in another molybdenum resistance heating boat; 100 mg of D-1 were charged in another molybdenum resistance heating boat; 200 mg of HE-1 were charged in another molybdenum resistance heating boat; and 200 mg of Alq₃ were further charged in another molybdenum resistance heating boat.

Next, after depressurizing the vacuum chamber to 4×10⁻⁴ Pa, the foregoing heating boat in which CuPc was charged was heated via electricity application to form a hole injection layer having a thickness of 20 nm on a transparent supporting substrate via evaporation at an evaporation rate of 0.1 nm/sec. Further, the foregoing heating boat in which α-NPD was charged was heated via electricity application to form a hole transport layer having a thickness of 50 nm on a transparent supporting substrate via evaporation at an evaporation rate of 0.1 nm/sec.

Further, the foregoing heating boat in which H-1 was charged, and the foregoing heating boat in which D-1 was charged were heated via electricity application to form an emission layer having a thickness of 30 nm on the foregoing hole transport layer via co-evaporation at an evaporation rate of 0.2 nm/sec and at an evaporation rate of 0.01 nm/sec, respectively.

Further, the foregoing heating boat in which HB-1 was charged was heated via electricity application to form a hole blocking layer having a thickness of 10 nm on an emission layer via evaporation at an evaporation rate of 0.1 nm/sec.

Further, the foregoing heating boat in which Alq₃ was charged was heated via electricity application to form an electron transport layer having a thickness of 40 nm on a hole blocking layer via evaporation at an evaporation rate of 0.1 nm/sec.

In addition, the substrate temperature during evaporation was room temperature. Subsequently, 0.5 nm thick lithium fluoride was prepared via evaporation as a cathode buffer layer, and 110 nm thick aluminum was further prepared via evaporation for a cathode to produce organic EL element 4-1.

Organic EL element 4-2 was prepared similarly to preparation of organic EL element 4-1, except that the foregoing heating boat in which H-1 was charged was heated via electricity application, and evaporated at an evaporation rate of 0.1 nm/sec to provide an intermediate layer having a thickness of 10 nm between a hole transport layer and an emission layer in organic EL element 4-1.

Organic EL element 4-3 was prepared similarly to preparation of organic EL element 4-1, except that dopant material D-1 was replaced by D-2, and organic EL element 4-4 was prepared similarly to preparation of organic EL element 4-2, except that dopant material D-1 was replaced by D-2.

Further, organic EL element 4-S was prepared similarly to preparation of organic EL element 4-1, except that the host material was replaced by H-29, and organic EL element 4-6 was prepared similarly to preparation of organic EL element 4-2, except that the host material was replaced by H-29, and the intermediate material was replaced by H-29.

Further, organic EL element 4-7 was prepared similarly to preparation of organic EL element 4-5, except that the dopant material was replaced by D-2, and organic EL element 4-8 was prepared similarly to preparation of organic EL element 4-6, except that the dopant material was replaced by D-2 (shown in Table 4).

However, the relationship between μ1 designated as hole mobility of hole transport material (α-NPD) and μ2 designated as hole mobility of intermediate material (H-1, H-29) was obtained via measurement employing TOF-301 manufactured by OPTEL Co., Ltd. to confirm μ1>μ2 from carrier arrival time (t) thereof.

Further, H-1 has an ionization potential of 6.1 eV and a triplet excitation energy of 3.0 eV, and D-1 has an ionization potential of 5.0 eV and a triplet excitation energy of 2.63 eV. H-1 and D-1 satisfy foregoing Formulae (2) and (3) since hole transport material α-NPD has an ionization potential of 5.5 eV. H-1 is also a material satisfying μe>μh.

Also in cases where H-29 was employed as a host material or an intermediate material, and D-2 was employed as a dopant material, these measurements of ionization potential results in satisfaction of foregoing Formulae (2) and (3). Further, μe as electron mobility of host material H-29 was larger than μh as the hole mobility via measurement with a Time-Of-Flight (T.O.F) method employing TOF-301 manufactured by OPTEL Co., Ltd.

### <<Evaluation of Organic EL Elements 4-1-4-8>>

The resulting organic EL elements 4-1-4-8 were evaluated similarly to evaluation of Example 1, and results thereof are shown in Table 4. The measured result of emission lifetime of organic EL element 4-2 was expressed by a relative value when the emission lifetime of organic EL element 4-1 was set to 100%. The measured result of emission lifetime of organic EL element 4-4 was also expressed by a relative value when the emission lifetime of organic EL element 4-3 was set to 100%. The measured result of emission lifetime of organic EL element 4-6 was further expressed by a relative value when the emission lifetime of organic EL element 4-5 was set to 100%. The measured result of emission lifetime of organic EL element 4-8 was further expressed by a relative value when the emission lifetime of organic EL element 4-7 was set to 100%.

TABLE 4

| | Inter- | | | Lifetime in | |
|---|---|---|---|---|---|
| | mediate | Host | Dopant | relative | |
| Element | layer | material | material | value (%) | |
| 4-1 | Not provided | H-1 | D-1 | 100 | Comparative |
| | | | | | example |
| 4-2 | H-1 | H-1 | D-1 | 500 | Present |
| | | | | | invention |
| 4-3 | Not provided | H-1 | D-2 | 100 | Comparative |
| | | | | | example |
| 4-4 | H-1 | H-1 | D-2 | 500 | Present |
| | | | | | invention |
| 4-5 | Not provided | H-29 | D-1 | 100 | Comparative |
| | | | | | example |
| 4-6 | H-29 | H-29 | D-1 | 300 | Present |
| | | | | | invention |
| 4-7 | Not provided | H-29 | D-2 | 100 | Comparative |
| | | | | | example |
| 4-8 | H-29 | H-29 | D-2 | 300 | Present |
| | | | | | invention |

As is clear from Table 4, it is to be understood that electron mobility of a host material is larger than the hole mobility, and the relationship between ionization potential and each of a hole transport material, a host material and a dopant material satisfies Formulae (2) and (3), even though each of the host material, the intermediate layer material and the dopant material is changed, and the organic EL element exhibits longer lifetime when an intermediate layer in which the relationship between hole mobility and the hole transport material satisfies Formula (5) is provided.

### Example 5

A substrate (NA45, produced by NH Techno Glass Corp.), prepared by forming a 100 nm thick ITO (indium tin oxide) film as an anode on a glass plate having a size of 100×100×1.1 mm, was subjected to patterning, and a transparent supporting substrate provided with this ITO transparent electrode was cleaned with isopropyl alcohol via ultrasonic waves, followed by being dried employing dry nitrogen gas and being cleaned for 5 minutes employing UV ozone. This transparent supporting substrate was fixed on a substrate holder in a commercially available vacuum evaporator, and installed in the vacuum evaporator. On the other hand, inside the vacuum evaporator, 200 mg of CuPc were charged in a molybdenum resistance heating boat; 200 mg of α-NPD were charged in another molybdenum resistance heating boat; 300 mg of H-1 were charged in another molybdenum resistance heating boat; 100 mg of D-1 were charged in another molybdenum resistance heating boat; 200 mg of HB-1 were charged in another molybdenum resistance heating boat; and 200 mg of Alq₃ were further charged in another molybdenum resistance heating boat.

Next, after depressurizing the vacuum chamber to 4×10⁻⁴ Pa, the foregoing heating boat in which CuPc was charged was heated via electricity application to form a hole injection layer having a thickness of 20 nm on a transparent supporting substrate via evaporation at an evaporation rate of 0.1 nm/sec. Further, the foregoing heating boat in which α-NPD was charged was heated via electricity application to form a hole transport layer having a thickness of 50 nm on a transparent supporting substrate via evaporation at an evaporation rate of 0.1 nm/sec.

Further, the foregoing heating boat in which H-1 was charged, and the foregoing heating boat in which Ir(ppy)₃ was charged were heated via electricity application to form an emission layer having a thickness of 30 nm on the foregoing hole transport layer via co-evaporation at an evaporation rate of 0.2 nm/sec and at an evaporation rate of 0.01 nm/sec, respectively.

Next, the foregoing heating boat in which H-1 was charged was heated via electricity application to form an intermediate layer having a thickness of 10 nm via evaporation at an evaporation rate of 0.1 nm/sec.

Further, the foregoing heating boat in which H-1 was charged, and the foregoing heating boat in which D-1 was charged were heated via electricity application to form an emission layer having a thickness of 30 nm on the foregoing hole transport layer via co-evaporation at an evaporation rate of 0.2 nm/sec and at an evaporation rate of 0.01 nm/sec, respectively.

Further, the foregoing heating boat in which HB-1 was charged was heated via electricity application to form a hole blocking layer having a thickness of 10 nm on an emission layer via evaporation at an evaporation rate of 0.1 nm/sec.

Further, the foregoing heating boat in which Alq₃ was charged was heated via electricity application to form an electron transport layer having a thickness of 40 nm on a hole blocking layer via evaporation at an evaporation rate of 0.1 nm/sec.

In addition, the substrate temperature during evaporation was room temperature. Subsequently, 0.5 nm thick lithium fluoride was prepared via evaporation as a cathode buffer layer, and 110 nm thick aluminum was further prepared via evaporation for a cathode to produce organic EL element 5-1.

Organic EL element 5-2 was prepared similarly to preparation of organic EL element 5-1, except that one in which no intermediate layer was provided was arranged to be set to intermediate layer H-1 in organic EL element 5-1.

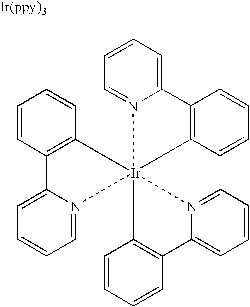

Incidentally, the combination selected from ionization potential of hole transport material (α-NPD), ionization potential of intermediate material (H-1) ionization potential of emission layer host material (H-1) and ionization potential of dopant material (D-1) is satisfied by foregoing Formulae (2) and (3). Further, μ1 as hole mobility of hole transport material (α-NPD) is larger than μ2 as hole mobility of intermediate material (H-1). Further, host material H-1 satisfies the following inequality of μe>μh. In addition, E4 as ionization potential of Ir(ppy)₃ measured via photoelectron spectroscopy was 5.6 eV.

### <<Evaluation of Organic EL Elements 5-1 and 5-2>>

The resulting organic EL elements 5-1 and 5-2 prepared as described in Example 5 were evaluated, and results thereof are shown in Table 5. The measured result of emission lifetime of organic EL element 5-2 was compared with that of organic EL element 5-1, and those were expressed by a relative value when the emission lifetime of organic EL element 3-3 was set to 100%.

TABLE 5

| | Anode side | Cathode side | Inter- | | | Lifetime in | |
|---|---|---|---|---|---|---|---|
| | emission | emission | mediate | | | relative | |
| *1 | layer Dopant | layer Host | layer | Host | Dopant | value (%) | |
| 5-1 | Ir (ppy) ₃ | H-1 | Not | H-1 | D-1 | 100 | Comp. |
| | | | provided | | | | |
| 5-2 | Ir (ppy) ₃ | H-1 | H-1 | H-1 | D-1 | 120 | Inv. |

*1: Element
Comp.: Comparative example
Inv.: Present invention

It is to be understood that the organic EL element of the present invention exhibits longer lifetime, when an intermediate layer having the relationship expressed by foregoing Formulae (2) and (3) is provided among host material (H-1) and dopant material (D-1) of the emission layer brought into contact with intermediate material (H-1), an intermediate layer and hole transport material (α-NPD) in cases where the intermediate layer brought into contact with the anode side of the emission layer is provided (even though another emission layer is provided on the anode side of the intermediate layer).

1-41. (canceled)

42. An organic electroluminescent element comprising an emission layer containing a host material and a dopant material, and a hole transport layer, provided between a cathode and an anode facing each other,
wherein an intermediate layer is provided on an anode side of the emission layer so as to be brought into contact with the emission layer, and the hole transport layer is provided on the anode side of the intermediate layer,
the organic electroluminescent element satisfying the following Formulae (2) and (3):
E1<E2≦E3 Formula (2)
E2>E4 Formula (3)
wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material.

43. The organic electroluminescent element of claim 42,
wherein the intermediate layer is provided on the anode side of the emission layer so as to be brought into contact with the emission layer, and the hole transport layer is provided on the anode side of the intermediate layer so as to be brought into contact with the intermediate layer,
the organic electroluminescent element satisfying the following Formula (1):
μe>μh Formula (1)
wherein μe and μh represent electron mobility and hole mobility of the host material, respectively, and
further satisfying the following Formulae (2) and (3):
E1<E2≦E3 Formula (2)
E2>E4 Formula (3)
wherein E1 represents an ionization potential of a hole transport material constituting the hole transport layer, E2 represents an ionization potential of an intermediate material constituting the intermediate layer, E3 represents an ionization potential of the host material, and E4 represents an ionization potential of the dopant material.

44. The organic electroluminescent element of claim 42,
wherein the dopant material produces phosphorescence.

45. The organic electroluminescent element of claim 44,
wherein the dopant material has a triplet excitation energy of at least 2.58 eV.

46. The organic electroluminescent element of claim 44,
wherein the dopant material has an ionization potential E4 of 5.3 eV or less.

47. The organic electroluminescent element of claim 44,
wherein the dopant material comprises a compound having a partial structure represented by the following Formula (1): 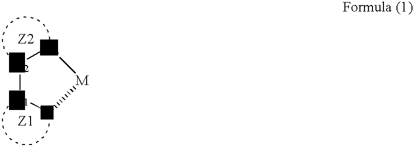
wherein X₁, X₂ and X₃ each represent a carbon atom or a nitrogen atom; Z1 represents a residue to form a 5-member aromatic heterocycle; Z2 represents a 6-member aromatic ring, a 5-member aromatic heterocycle or a 6-member aromatic heterocycle; and M represents Ir or Pt.

48. The organic electroluminescent element of claim 47,
wherein the dopant material comprises a compound having a partial structure represented by the following Formula (2): 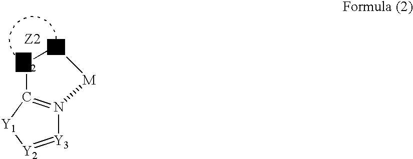
wherein X₂ and X₃ each represent a carbon atom or a nitrogen atom; Y₁ represents NR₁, O or S; Y₂ and Y₃ each represent a carbon atom or a nitrogen atom; Z2 represents a 6-member aromatic ring, a 5-member aromatic heterocycle or a 6-member aromatic heterocycle; M represents Ir or Pt; and R₁ represents a hydrogen atom, an aliphatic group, an aromatic group or a heterocyclic group.

49. The organic electroluminescent element of claim 44,
wherein the dopant material comprises a compound having a partial structure represented by the following Formula (6): 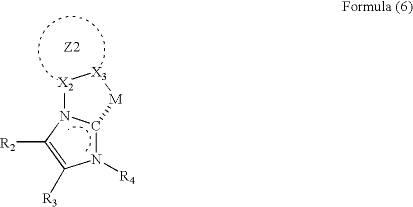
wherein X₂ and X₃ each represent a carbon atom or a nitrogen atom; R₂, R₃ and R₄ each represent a hydrogen atom or a substituent; Z2 represents a 6-member aromatic ring, a 5-member aromatic heterocycle or a 6-member aromatic heterocycle; and M represents Ir or Pt.

50. The organic electroluminescent element of claim 42,
wherein the intermediate material has a triplet excitation energy of at least 2.58 eV.

51. The organic electroluminescent element of claim 50,
wherein the intermediate material and the host material are the same in compound.

52. The organic electroluminescent element of claim 42,
wherein the intermediate layer has a thickness of 1-20 nm.

53. The organic electroluminescent element of claim 42, satisfying the following Formula (4):
0.001<L2/(L1+L2+L3)<0.2 Formula (4)
wherein L1 represents thickness of the hole transport layer, L2 represents thickness of the intermediate layer, and L3 represents thickness of the emission layer.

54. The organic electroluminescent element of claim 43,
wherein the host material comprises any one of a carbazole ring, a carboline ring and a triaryl amine structure.

55. The organic electroluminescent element of claim 42, satisfying the following Formula (5):
μ1>μ2 Formula (5)
wherein μ1 represents hole mobility of the hole transport material, and μ2 represents hole mobility of an intermediate material constituting the intermediate layer.

56. The organic electroluminescent element of claim 42, comprising another emission layer between the intermediate layer and the hole transport layer.

57. A lighting device comprising the organic electroluminescent element of claim 42.

58. A display device comprising the organic electroluminescent element of claim 42.
